(19) United States
(12) Patent Application Publication
(10) Pub. No.: US 2011/0054012 A1
(43) Pub. Date: Mar. 3, 2011

(54) Methods and Compositions for Increasing Gene Expression

(76) Inventors: Robert F. Place, San Francisco, CA (US); Long-Cheng Li, San Francisco, CA (US); Rajvir Dahiya, San Francisco, CA (US)

(21) Appl. No.: 12/810,749

(22) PCT Filed: Dec. 23, 2008
(86) PCT No.: PCT/US08/88243

§ 371 Date: Sep. 9, 2010

Related U.S. Application Data
(60) Provisional application No. 61/017,449, filed on Dec. 28, 2007.
(60) Provisional application No. 61/023,793, filed on Jan. 25, 2008.

(51) Int. Cl. A61K 31/7105; C12N 5/071; C07H 21/02; A61P 35/00; C12N 15/63; A61K 31/713

(52) U.S. Cl. 514/44 R; 435/375; 536/23.2; 435/320.1

(57) ABSTRACT

The present invention provides compositions, pharmaceutical preparations, kits and methods for increasing expression of a gene product in a cell by contacting the cell with a microRNA (miRNA) molecule comprising a ribonucleic strand that is complementary to a non-coding nucleic acid sequence of the gene.

### CROSS-REFERENCE

This application claims the benefit of U.S. Provisional Application Nos. 61/017,449, filed Dec. 28, 2007, and 61/023,793, filed Jan. 25, 2008, which applications are incorporated herein by reference.

### GOVERNMENT RIGHTS

This invention was made with government support under federal grant nos. R01CA101844, R01CA111470, T32DK007790, and R21CA131774 awarded by National Institutes of Health and PC073790 awarded by the Department of Defense. The United States Government has certain rights in this invention.

### BACKGROUND OF THE INVENTION

Small double-stranded RNAs (dsRNAs) are known to be the trigger of RNA interference (RNAi) (Fire et al., (1998) Nature 391: 806-11; Elbashir et al., (2001) Nature 411: 494-8.) MicroRNAs (miRNAs) are a group of small non-coding RNAs that serve as endogenous sources of dsRNA. In a manner similar to RNAi, cells utilize miRNAs to negatively regulate gene expression by repressing translation or directing sequence-specific degradation of target mRNAs (Zeng et al., (2003) Proc Natl Aced Sci USA 100: 9779-84; Lee et al., (1993) Cell 75, 843-54). In this regard, miRNAs are considered to be key regulators of gene expression.

It is currently believed that miRNAs elicit their effect by silencing the expression of target genes (He et al., (2004) Nat Rev Genet. 5, 522-31). MicroRNAs (miRNA) play important roles in numerous cellular processes including development, proliferation, and apoptosis (Carrington et al., (2003) Science 301, 336-8). Cancer development has also been linked to alterations in miRNA expression patterns (Lu et al., (2005) Nature 435, 834-8, O'Donnell et al., (2005) Nature 435, 839-43, He et al., (2005) Nature 435, 828-33).

It was previously reported that synthetic dsRNAs targeting promoter regions induce gene expression; a phenomenon referred to as RNA activation (RNAa) (Li et al., (2006) Proc Natl Acad Sci USA 103, 17337-42. Others have since observed similar results (Janowski, et al., (2007) Nat Chem Biol 3, 166-73). RNAa induces predictable changes in phenotype and affects downstream gene expression in response to targeted gene induction (Li et al., supra; Janowski et al., supra). Much like RNAi, RNAa can manipulate gene expression to alter cellular pathways and change cell physiology. In a manner similar to RNAa, miRNAs may also function to positively regulate gene expression. By scanning gene promoters for sequences highly complementary to miRNAs, target sites for a particular miRNA, miR-373, were discovered in the promoters of E-cadherin and CSDC2 (cold shock domain containing protein C2). It was discovered that miR-373 induced robust expression of both E-cadherin and CSDC2, thus identifying a new potential function for miRNA in gene activation.

Generally, application of miRNA has been limited to gene silencing or reduction of gene expression and has not been applied to gene activation. There is accordingly still a need for compounds that can activate gene expression, and methods of using such compounds for the study and treatment of genetic disorders. The present invention addresses these needs, as well as others.

### SUMMARY OF THE INVENTION

The present invention provides compositions, pharmaceutical preparations, kits and methods for increasing expression of a gene product in a cell by contacting the cell with a miRNA molecule comprising a ribonucleic strand that is complementary to a non-coding nucleic acid sequence of the gene.

These and other advantages of the invention will be apparent from the detailed description below.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1, panel A—Is a schematic representation of the E-cadherin promoter and its CpG island. Indicated is the location of the miR-373 target site. FIG. 1, panel B—is an illustration of the sequence of the miR-373 target site located at minus (−)645 nucleotides in the 5′ direction relative to the transcription start site. Bases in bold indicate the target site in the sense strand of promoter DNA. Sequences complementary to the target site are indicated by a solid dash. Sequences in the target site where G:U/T wobble base-pairing occur between miR-373 and E-Cadherin are indicated by a colon. The upper sequence is the wild type promoter sequence of the E-cadherin gene (SEQ ID NO:1). The bottom sequence is miR-373AS (SEQ ID NO:3). FIG. 1, panel C—Shows sequence and structure of miR-373 and dsEcad-640. Lower case letters in both strands of miR-373 correspond to the native two base 3′ overhangs. Sequences complementary to the target site are shown by a dash. Sequences where G:U/T wobble base pairing occur between the miR-373 ribonucleic acid strands are indicated by a colon. The sequence on the left side of the page, top strand is miR-373 S (SEQ ID NO:2). The sequence on left side of the page, bottom strand is miR-373AS (SEQ ID NO:3). As used herein when referring to sequences of the invention, “S” refers to the sense strand, and “AS” refers to the anti-sense strand. The sequence on the right side of the page, top strand is dsEcad-6405 (SEQ ID NO:4). The sequence on the right side of the page, bottom strand is dsEcad-640AS (SEQ ID NO:5). FIG. 1, panel D—Is a gel photograph showing E-cadherin expression levels after PC-3 cells were transfected with the indicated dsRNAs. Combination treatment of miR-373 and dsEcad-640 (miR-373+dsEcad-640) was performed using equal quantities of each dsRNA. Mock samples were transfected in the absence of dsRNA. E-cadherin and GAPDH mRNA expression levels were assessed by standard RT-PCR with GAPDH as a loading control. FIG. 1, panel E—Is a graphical representation showing relative expression of E-cadherin determined by real-time PCR (mean±standard error from 4 independent experiments). Values of E-cadherin were normalized to GAPDH. FIG. 1, panel F—Is a Western blot showing induction of E-cadherin polypeptide. GAPDH was also detected and served as a loading control.

FIG. 2, panel A—Shows an illustration of the sequence of the miR-373 precursor hairpin RNA (Pre-miR-373). Pre-miR-373 is a 61 nucleotide RNA molecule that forms a hairpin like structure (SEQ ID NO:14). FIG. 2, panel B—Is a gel photograph showing E-cadherin expression levels after PC-3 cells were transfected with pre-miR-Con, pre-miR-373, or miR-373. E-cadherin and GAPDH mRNA expression levels were assessed by standard RT-PCR. FIG. 2, panel C—Is a graphical representation of relative expression was determined by real-time PCR (mean±standard error from 4 independent experiments). Values of E-cadherin were normalized to GAPDH. FIG. 2, panel D—Is a Western blot showing E-cadherin and GAPDH polypeptide levels. GAPDH served as a loading control.

FIG. 3, panel A is a graphical representation of cellular levels of miR-373 showing that pre-miR373 is processed into miR-373 and both can be detected at the same level. FIG. 3, panel B shows the relative level of endogenous miR-373 in respective cell lines.

FIG. 4, panel A—Is a Western blot illustrating the effect on Dicer after PC-3 cells were treated with Dicer-PMO (phosphorodiamidate morpholino oligonucleotide) or Con-PMO (control). Cells were transfected with miR-373 or pre-miR-373 following initial PMO treatments. Total protein was extracted and levels of Dicer and GAPDH were determined by immunoblot analysis. GAPDH served as a loading control. FIG. 4, panel B—Is a photograph of a gel showing E-cadherin expression levels after PC-3 cells were treated with or without Dicer-PMO or Con-PMO. The following day, cells were transfected with pre-miR-Con or premiR-373. E-cadherin and GAPDH mRNA expression levels were assessed by standard RT-PCR. FIG. 4, panel C—Is a photograph of a gel showing E-cadherin levels after PC-3 cells were treated with the indicated PMO molecules. The following day, cells were transfected with dsControl or miR-373. E-cadherin and GAPDH mRNA expression levels were assessed by standard RT-PCR.

FIG. 5, panel A—Is an illustration of the sequence of the miR-373 target site located at minus (−)787 nucleotides in the 5′ direction relative to the transcription start site in the CSDC2 promoter. Bases in bold indicate the putative target site in the sense strand of promoter DNA. The bases of miR-373 that are complementary to the target site are shown by a dash. The bases of miR-373 where G:U/T wobble base-pairing occur between miR-373 and the CSDC2 target sequence are shown by a colon. The upper sequence is the wild type sequence of the CSDC2 promoter region (SEQ ID NO:28). The bottom strand is miR-373 AS (SEQ ID NO:3). FIG. 5, panel B—Is a gel showing CSDC2 expression levels after PC-3 cells were transfected with dsControl or miR-373. Expression of CSDC2 and GAPDH was determined by standard RT-PCR. GAPDH served as a loading control. FIG. 5, panel C—Is a photograph of a gel showing CSDC2 and GAPDH expression levels in PC-3 cells following mock, premiR-Con or pre-miR-373 transfections. FIG. 5, panel D—Is a graphical representation showing relative expression of CSDC2 determined by real-time PCR (mean±standard error from 4 independent experiments). Values of CSDC2 were normalized to GAPDH.

FIG. 6 is photograph of a gel depicting enrichment of RNApII at miR-373-targeted promoters. PC-3 cells were transfected with mock, dsControl, or miR-373. ChIP assays were performed using an RNApII-specific antibody to immunoprecipitate transcriptionally active regions of DNA. The absence of antibody (No AB) served to identify background amplification. Input DNA was amplified as a loading control.

FIG. 7, panel A—Is a graphical depiction of sequence showing mutations to 4 of the first or last nucleotides in miR-373 resulting in miR-373-5MM and miR-373-3MM, respectively. The mutated bases are shown in bold. Panel A top sequence, top strand is miR-3735 (SEQ ID NO:2), Panel A top sequence, bottom strand is miR-373AS (SEQ ID NO:3). Panel A center sequence, top strand is miR-373-5MM S (SEQ ID NO:6). Panel A, center sequence, bottom strand is miR-373-5MM AS (SEQ ID NO:7). Panel A, bottom sequence, top strand is miR-373-3MM S (SEQ ID NO:8). Panel A, bottom sequence, bottom strand is miR-373-3MM AS (SEQ ID NO:9). FIG. 7, panel B—Is a gel photograph showing E-cadherin and CSDC2 expression after cells were transfected with each indicated miRNA duplex. GAPDH served as a loading control. FIG. 7, panel C—Is a photograph of a gel showing miRNAs targeting specific sites in either the E-cadherin (dsEcad-215) or CSDC2 (dsCSDC2-670) promoter specifically induce the expression of only the targeted gene.

FIG. 8, panel A is a photograph of a gel showing that anti-miR-373 inhibits miR-373-induced gene expression. FIG. 8, panel A shows the transcript levels of CSDC2 and E-cadherin after cells were transfected with miR-373 in combination with anti-miR-Con or anti-miR-373. FIG. 8, panel B shows transcript levels of CSDC2 and E-cadherin after cells were co-transfected with pre-miR-373 and anti-miR-Con or anti-miR-373. GAPDH served as a loading control.

FIG. 9, panel A is a photograph of a gel showing that a combination of miR-373 and dsEcad-215 transfected into PC-3 cells together may additively increase E-cadherin levels. GAPDH served as a loading control. FIG. 9, panel B is a graphical representation of relative expression of E-cadherin determined by real-time PCR, showing the additive increase of two miRNAs, producing approximately double the expression level of dsEcad-215.

FIG. 10 shows that dsRNAs targeting the mouse Ccnb1 gene promoter induce Ccnb1 gene expression. Panel A shows a schematic representation of the dsRNA location in the Ccnb1 promoter relative to the transcriptional start site. Panel B shows NIH/3T3 cells that were transfected with 50 nM dscontrol or dsCcnb1 for 72 hrs. mRNA expression of Ccnb1 was analyzed by real-time RT-PCR and normalized to that of β-actin. Mock samples were transfected in the absence of dsRNA. Data represents mean±SE of three independent experiments. Panel C shows NIH/3T3 cells that were transfected as in Panel B and the protein level of Ccnb1 was examined by western blotting analysis.

FIG. 11 shows that miRNAs targeting the mouse Ccnb1 gene promoter induce Ccnb1 gene expression. Panel A shows a schematic representation of the miRNA location in the Ccnb1 promoter relative to the transcriptional start site. Panel B shows NIH/3T3 cells that were transfected with 30 nM pre-miRNA control, miR-744, or miR-1186 for 72 hrs. mRNA expression of Ccnb1 was analyzed by real-time RT-PCR and normalized to that of β-actin. Data represents mean±SE of three independent experiments. Panel C shows NIH/3T3 cells that were transfected as in Panel B and the protein level of Ccnb1 was examined by western blotting analysis. Panels D and E show NIH/3T3 cells that were transfected with 50 nM control sRNA or siRNA against Dicer (Panel D) and Drosha (Panel E). The knockdown efficiency was examined by real-time RT-PCR. Panel F shows the effect of knockdown of Drosha and Dicer on Ccnb1 gene expression was analyzed by real-time RT-PCR.

FIG. 12 shows that Ago1 mediates transcriptional activation of Ccnb1 in NIH/3T3 cells. Panel A shows establishment of stable NIH/3T3 cell lines overexpressing Ago1 or EGFP. Both Ago1 and EGFP were cloned to possess N-terminal HA-epitope tags. Stable overexpression of Ago1 or EGFP was confirmed by immunoblot analysis using an antibody specific to the HA-epitope tag. GAPDH severed as a loading control. Quantitative real-time PCR was used to measure Ago1 levels at the indicated promoter sites. Ago1 enrichment was determined by dividing the levels of Ago1 in the presence of the HA antibody (+HA) by the background levels in the no antibody control (—HA). Panel B shows real-RT PCR analysis of Ccnb1 gene expression in EGFP- and Ago1-NIH/3T3 cell lines. Expression level was normalized to β-actin. Data represents mean±SE of three biological samples. Panels C and D show Ago1 was knocked down in NIH/3T3 cells using sRNA. Expression of Ago1 (Panel C) and Ccnb1 (Panel D) in Ago1 depleted NIH/3T3 cells was analyzed by real-time PCR.

FIG. 13 shows that Ago1 associates with the proximal promoter region of mouse Ccnb1. ChIP experiments were performed with anti-HA antibody in NIH3T3-Ago1 and NIH3T3-EGFP cells to examine Ago1-DNA interactions. Panel A shows a schematic illustration of the primers used for scanning Ago1 binding in the Ccnb1 2-kb proximal promoter region. Starting positions of the forward primers relative to the transcription start site are shown. Panel B shows a fold enrichment by Ago1 relative to negative control (ACTB and GAPDH) was determined by quantitative PCR. Data were normalized to input DNA. Error bars represent S.E.M. for three independent experiments.

### DETAILED DESCRIPTION OF THE INVENTION

The present invention provides compositions, pharmaceutical preparations, and methods for increasing activity of a gene product through transcriptional activation of the encoding gene in a cell by contacting the cell with a miRNA molecule comprising a ribonucleic strand that is complementary to a non-coding nucleic acid sequence of the gene. Also provided are kits for practicing the subject methods of the invention.

Before the present invention described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a sample” includes a plurality of such samples and reference to “the molecule” includes reference to one or more molecules and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

As used herein the term “isolated” is meant to describe a compound of interest (e.g., either a polynucleotide or a polypeptide) that is in an environment different from that in which the compound might naturally occur.

“Purified” as used herein refers to a compound removed from an environment in which it was produced and is at least 60% free, preferably 75% free, and most preferably 90% free from other components with which it is naturally associated or with which it was otherwise associated with during production.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. “Perfect complementarity” or “100% complementarity” refers to the situation in which each nucleotide unit of one polynucleotide strand can hydrogen bond with a nucleotide unit of a second polynucleotide strand, without a “mismatch.” Less than perfect complementarity refers to the situation in which not all nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. Substantial complementarity refers to about 79%, about 80%, about 85%, about 90%, about 95%, or greater complementarity. Thus, for example, two polynucleotides of 29 nucleotide units each, wherein each comprises a di-dT at the 3′ terminus such that the duplex region spans 27 bases, and wherein 26 of the 27 bases of the duplex region on each strand are complementary, are substantially complementary since they are 96.3% complementary when excluding the di-dT overhangs. In determining complementarity, overhang regions are excluded.

The term “conjugate” refers to a polynucleotide that is covalently or non-covalently associated with a molecule or moiety that alters the physical properties of the polynucleotide such as increasing stability and/or facilitate cellular uptake of miRNA by itself. A “terminal conjugate” may have a molecule or moiety attached directly or indirectly through a linker to a 3′ and/or 5′ end of a polynucleotide or double stranded polynucleotide. An internal conjugate may have a molecule or moiety attached directly or indirectly through a linker to a base, to the 2′ position of the ribose, or to other positions that do not interfere with Watson-Crick base pairing, for example, 5-aminoallyl uridine.

In a double stranded polynucleotide, one or both 5′ ends of the strands of polynucleotides comprising the double stranded polynucleotide can bear a conjugated molecule or moiety, and/or one or both 3′ ends of the strands of polynucleotides comprising the double stranded polynucleotide can bear a conjugated molecule or moiety.

Conjugates may contain, for example, amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates are steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Still other examples include thioethers such as hexyl-S-tritylthiol, thiocholesterol, acyl chains such as dodecandiol or undecyl groups, phospholipids such as di-hexadecyl-rac-glycerol, triethylammonium 1,2-di-O-hexadecyl-rac-glycer-o-3-H-phosphonate, polyamines, polyethylene glycol, adamantane acetic acid, palmityl moieties, octadecylamine moieties, hexylaminocarbonyl-oxyc-holesterol, farnesyl, geranyl and geranylgeranyl moieties.

Conjugates can also comprise a detectable label. For example, conjugates can be a polynucleotide covalently attached to a fluorophore. Conjugates may include fluorophores such as TAMRA, BODIPY, Cyanine derivatives such as Cy3 or Cy5, Dabsyl, or any other suitable fluorophore known in the art.

A conjugate molecule or moiety may be attached to any position on the terminal nucleotide that is convenient and that does not substantially interfere with the desired activity of the polynucleotide(s) that bear it, for example the 3′ or 5′ position of a ribosyl sugar. A conjugate molecule or moiety substantially interferes with the desired activity of a miRNA if it adversely affects its functionality such that the ability of the miRNA to mediate gene activation is reduced by greater than 80% in an in vitro assay employing cultured cells, where the functionality is measured at 24 hours post transfection.

The phrase or “effective concentration” refers to a concentration of miRNA in a cell effective to cause an increase in transcription of a gene of interest in the cell. Of particular interest is an effective concentration that provides a greater than or equal to at least about 45% or more increase, including about 50% or more, about 60% or more, about 70% or more, about 75% or more, about 80% or more increase in target sequence activity relative to a basal expression level. Target sequence activity may be measured by any method known in the art. For example, where the target sequence is a promoter, target sequence activity may be measured by level of transcription, level of the protein whose transcription is operably linked or operably associated with the promoter, or activity of the protein whose transcription is operably linked or operably associated with the promoter, or by detection of a marker gene, for example, lacZ, one of the family of fluorescent polypeptides (e.g., GFP, YFP, BFP, RFP etc), or luciferase which is operably linked to the promoter.

The term “polynucleotide” refers to polymers of nucleotides, and includes but is not limited to single stranded or double stranded molecule of DNA, RNA, or DNA/RNA hybrids including polynucleotide chains of regularly and irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides wherein the substitution or attachment of various entities or moieties to the nucleotide units at any position, as well as naturally-occurring or non-naturally occurring backbones, are included.

The term “polyribonucleotide” refers to a polynucleotide comprising two or more modified or unmodified ribonucleotides and/or their analogs.

The term “ribonucleotide” and the phrase “ribonucleic acid” (RNA), refer to a naturally occurring or non-naturally occurring (artificial, synthetic), modified or unmodified nucleotide or polynucleotide. A ribonucleotide unit comprises an oxygen attached to the 2′ position of a ribosyl moiety that has a nitrogenous base attached in N-glycosidic linkage at the 1′ position of a ribosyl moiety, and a moiety that either allows for linkage to another nucleotide or precludes linkage. “Ribonucleic acid” as used herein can have a naturally occurring or modified phosphate backbone (e.g., as produced by synthetic techniques), and can include naturally-occurring or non-naturally-occurring, genetically encodable or non-genetically encodable, residues.

The term “deoxyribonucleotide” refers to a nucleotide or polynucleotide lacking an OH group at the 2′ and/or 3′ position of a sugar moiety. Instead it has a hydrogen bond to the 2′ and/or 3′ carbon.

“Deoxyribonucleic acid” as used herein can have a naturally occurring or modified phosphate backbone (e.g., as produced by synthetic techniques), and can include naturally-occurring or non-naturally-occurring, genetically encodable or non-genetically encodable, residues.

The term “gene” as used herein includes sequences of nucleic acids that when present in an appropriate host cell facilitates production of a gene product. “Genes” can include nucleic acid sequences that encode proteins, and sequences that do not encode proteins (e.g. promoters or enhancers), and includes genes that are endogenous to a host cell or are completely or partially recombinant (e.g., due to introduction of a exogenous polynucleotide encoding a promoter and a coding sequence, or introduction of a heterologous promoter adjacent an endogenous coding sequence, into a host cell). For example, the term “gene” includes nucleic acid that can be composed of exons and introns. Sequences that code for proteins are, for example, sequences that are contained within exons in an open reading frame between a start codon and a stop codon. “Gene” as used herein can refer to a nucleic acid that includes, for example, regulatory sequences such as promoters, enhancers and all other sequences known in the art that control the transcription, expression, or activity of another gene, whether the other gene comprises coding sequences or non-coding sequences. In one context, for example, “gene” may be used to describe a functional nucleic acid comprising regulatory sequences such as promoter or enhancer. The expression of a recombinant gene may be controlled by one or more heterologous regulatory sequences. “Heterologous” refers to two elements that are not normally associated in nature.

A “target gene” is a nucleic acid containing a sequence, such as, for example, a promoter or enhancer, against which an miRNA can be directed for the purpose of effectuating activation of expression. Either or both “gene” and “target gene” may be nucleic acid sequences naturally occurring in an organism, transgenes, viral or bacterial sequences, chromosomal or extrachromosomal, and/or transiently or chronically transfected or incorporated into the cell and/or its chromatin. A “target gene” can, upon miRNA-mediated activation, repress the activity of another “gene” such as a gene coding for a protein (as measured by transcription, translation, expression, or presence or activity of the gene's protein product). In another example, a “target gene” can comprise an enhancer, and miRNA mediated activation of the enhancer may increase the functionality of an operably linked or operably associated promoter, and thus increase the activity of another “gene” such as a gene coding for a protein that is operably linked to the increased promoter and/or enhancer.

“Regulatory elements” are nucleic acid sequences that regulate, induce, repress, or otherwise mediate the transcription, translation of a protein or RNA coded by a nucleic acid sequence with which they are operably linked or operably associated. Typically, a regulatory element or sequence such as, for example, an enhancer or repressor sequence, is operatively linked or operatively associated with a protein or RNA coding nucleic acid sequence if the regulatory element or regulatory sequence mediates the level of transcription, translation or expression of the protein coding nucleic acid sequence in response to the presence or absence of one or more regulatory factors that control transcription, translation and/or expression. Regulatory factors include, for example, transcription factors. Regulatory sequences may be found in introns.

Regulatory sequences or element include, for example, “TATAA” boxes, “CAAT” boxes, differentiation-specific elements, cAMP binding protein response elements, sterol regulatory elements, serum response elements, glucocorticoid response elements, transcription factor binding elements such as, for example, SPI binding elements, and the like. A “CAAT” box is typically located upstream (in the 5′ direction) from the start codon of a eukaryotic nucleic acid sequence encoding a protein or RNA. Examples of other regulatory sequences include splicing signals, polyadenylation signals, termination signals, and the like. Further examples of nucleic acid sequences that comprise regulatory sequences include the long terminal repeats of the Rous sarcoma virus and other retroviruses. An example of a regulatory sequence that controls tissue-specific transcription is the interferon-epsilon regulatory sequence that preferentially induces production of the operably linked sequence encoding the protein in placental, tracheal, and uterine tissues, as opposed to lung, brain, liver, kidney, spleen, thymus, prostate, testis, ovary, small intestine, and pancreatic tissues. Many, many regulatory sequences are known in the art, and the foregoing is merely illustrative of a few.

The term “enhancer” and phrase “enhancer sequence” refer to a variety of regulatory sequence that can increase the efficiency of transcription, without regard to the orientation of the enhancer sequence or its distance or position in space from the promoter, transcription start site, or first codon of the nucleic acid sequence encoding a protein with which the enhancer is operably linked or associated.

The term “promoter” refers to a nucleic acid sequence that does not code for a protein, and that is operably linked or operably associated to a protein coding or RNA coding nucleic acid sequence such that the transcription of the operably linked or operably associated protein coding or RNA coding nucleic acid sequence is controlled by the promoter. Generally, eukaryotic promoters comprise between 100 and 5,000 base pairs, although this length range is not meant to be limiting with respect to the term “promoter” as used herein. Although typically found 5′ to the protein coding nucleic acid sequence to which they are operably linked or operably associated, promoters can be found in intronic sequences as well.

The term “promoter” is meant to include regulatory sequences operably linked or operably associated with the same protein or RNA encoding sequence that is operably linked or operably associated with the promoter. Promoters can comprise many elements, including regulatory elements.

The term “promoter” comprises promoters that are inducible, wherein the transcription of the operably linked nucleic acid sequence encoding the protein is increased in response to an inducing agent. The term “promoter” may also comprise promoters that are constitutive, or not regulated by an inducing agent.

The phrases “operably associated” and “operably linked” refer to functionally related nucleic acid sequences. By way of example, a regulatory sequence is operably linked or operably associated with a protein encoding nucleic acid sequence if the regulatory sequence can exert an effect on the expression of the encoded protein. In another example, a promoter is operably linked or operably associated with a protein encoding nucleic acid sequence if the promoter controls the transcription of the encoded protein. While operably associated or operably linked nucleic acid sequences can be contiguous with the nucleic acid sequence that they control, the phrases “operably associated” and “operably linked” are not meant to be limited to those situations in which the regulatory sequences are contiguous with the nucleic acid sequences they control.

The phrase “non-coding target sequence” or “non-coding nucleic acid sequence” refers to a nucleic acid sequence of interest that is not contained within an exon or is a regulatory sequence.

The term “nucleotide” refers to a ribonucleotide or a deoxyribonucleotide or an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

“Nucleotide analogs” include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH2, NHR, NR2, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

“Modified bases” refer to nucleotide bases such as, for example, adenine, guanine, cytosine, thymine, and uracil, xanthine, inosine, and queuosine that have been modified by the replacement or addition of one or more atoms or groups. Some examples of types of modifications that can comprise nucleotides that are modified with respect to the base moieties, include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Further, the term “nucleotide” also includes those species that have a detectable label, such as for example a radioactive or fluorescent moiety, or mass label attached to the nucleotide.

The term “stabilized” refers to the ability of a miRNA to resist degradation while maintaining functionality and can be measured in terms of its half-life in the presence of, for example, biological materials such as serum. The half-life of an miRNA in, for example, serum refers to the time taken for the 50% of miRNA to be degraded.

The phrase “duplex region” refers to the region in two complementary or substantially complementary polynucleotides that form base pairs with one another, either by Watson-Crick base pairing or any other manner that allows for a duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” consists of 19 base pairs. The remaining base pairs may, for example, exist as 5′ and 3′ overhangs. Further, within the duplex region, 100% complementarity is not required; substantial complementarity is allowable within a duplex region. Substantial complementarity generally refers to about at least 79%, about 80%, about 85%, about 85%, about 90%, about 95% or greater complementarity. For example, a mismatch in a duplex region consisting of 19 base pairs (i.e., 18 base pairs and one mismatch) results in about 94.7% complementarity, rendering the duplex region substantially complementary. In another example, three mismatches in a duplex region consisting of 19 base pairs (i.e., 16 base pairs and three mismatches) results in about 84.2% complementarity, rendering the duplex region substantially complementary, and so on.

The term “overhang” refers to a terminal (5′ or 3′) non-base pairing nucleotide resulting from one strand extending beyond the other strand within a doubled stranded polynucleotide. One or both of two polynucleotides that are capable of forming a duplex through hydrogen bonding of base pairs may have a 5′ and/or 3′ end that extends beyond the 3′ and/or 5′ end of complementarity shared by the two polynucleotides. The single-stranded region extending beyond the 3′ and/or 5′ end of the duplex is referred to as an overhang.

The phrase “gene silencing” refers to the reduction in transcription, translation or expression or activity of a nucleic acid, as measured by transcription level, mRNA level, enzymatic activity, methylation state, chromatin state or configuration, translational level, or other measure of its activity or state in a cell or biological system. Such activities or states can be assayed directly or indirectly. “Gene silencing” refers to the reduction or amelioration of activity associated with a nucleic acid sequence, such as its ability to function as a regulatory sequence, its ability to be transcribed, its ability to be translated and result in expression of a protein, regardless of the mechanism whereby such silencing occurs.

As used herein, the terms “gene activating”, “activating a gene”, or “gene activation” are interchangeable and refer to an increase in transcription, translation or expression or activity of a nucleic acid, as measured by transcription level, mRNA level, enzymatic activity, methylation state, chromatin state or configuration, translational level, or other measure of its activity or state in a cell or biological system. Such activities or states can be assayed directly or indirectly. Furthermore, “gene activating”, “activating a gene”, or “gene activation” refer to the increase of activity associated with a nucleic acid sequence, such as its ability to function as a regulatory sequence, its ability to be transcribed, its ability to be translated and result in expression of a protein, regardless of the mechanism whereby such activation occurs.

The phrase “RNA interference” and the term “RNAi” refer to the process by which a polynucleotide or double stranded polynucleotide comprising at least one ribonucleotide unit exerts an effect on a biological process through disruption of gene expression. The process includes but is not limited to gene silencing by degrading mRNA, miRNA, interactions with tRNA, rRNA, hnRNA, cDNA and genomic DNA, as well as methylation of DNA and ancillary proteins.

The term “siRNA” and the phrase “short interfering RNA” refer to a double stranded nucleic acid that is capable of performing RNAi and that is between 18 and 30 base pairs in length (i.e., a duplex region of between 18 and 30 base pairs). Additionally, the term siRNA and the phrase “short interfering RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides. Generally, siRNA has high complementarity (generally 100%) in the ribonucleic acid strands that make up the duplex of the siRNA. In addition, generally the siRNA is highly complementary to the target sequence.

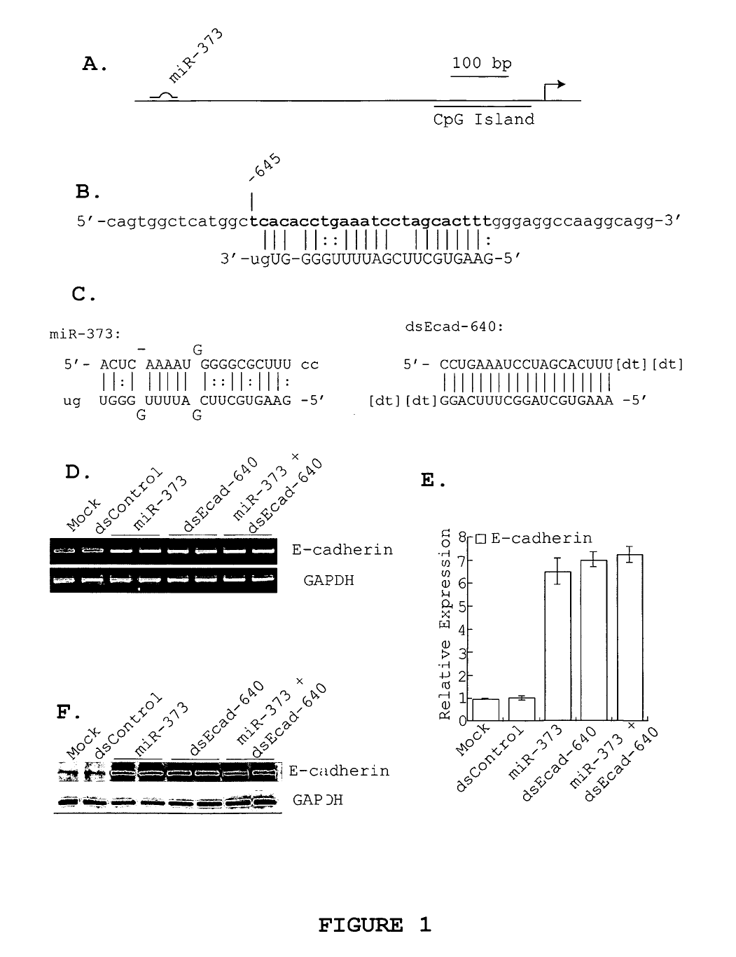

The term “pri-miRNA” as used herein refers to a precursor miRNA. In certain cases, pri-miRNAs are transcribed from a gene as primary RNA transcripts with a poly A tail and a 5′ cap. The pri-miRNAs are processed in the nucleus by the RNAase III-type enzyme Drosha into an intermediate form herein referred to as a “pre-miRNA.” A “pre-miRNA” as used herein is a duplex of ribonucleotide strands with a hairpin structure and may contain G:U/T wobble base pairing and bulges in secondary structure, which are discussed below. After processing the pre-miRNA is exported to the cytoplasm by the molecule Exportin-5. The pre-miRNA is processed by the RNAase III enzyme Dicer which produces a duplex MicroRNA or miRNA. The miRNA duplex is separated, with one strand becoming the miRNA and the other becoming degraded. As used herein, “miRNA” may refer to either the duplex form or a single stranded form. In certain cases, the miRNA has a sequence length of 19-27 nucleotides. For example, the miRNA miR373 has a length of 23 nucleotides. miRNAs contain G:U/T wobble base-pairing which allows non-complementarity between the individual ribonucleic strands of the miRNA. For example, this is shown in FIG. 1, panel C, where G:U/T base pairing occurs between the individual strands of miR-373 and is shown by a colon. miRNAs may also contain bulges in their secondary structure. This occurs when there is a region of non-complementarity between the individual ribonucleic acid strands. For example, in FIG. 1, panel C, the individual ribonucleic acid strands of miR-373 are non-complementary between guanine (G) bases, producing a bulge in the secondary structure of miR-373 (shown as a “G” base above and below the strand). In addition, miRNAs may provide for G:U/T wobble base pairing between the miRNA and the target sequence. For example, this is shown in FIG. 1, panel B, where G:U/T wobble base pairing between miR-373 and the E-Cadherin sequence is shown by a colon. The interaction between miRNA and the target sequence also allows for bulges in the secondary structure. For example, in FIG. 1, panel B, the region that does not show a dash or colon is non-complementary. This region of non-complementarity will create a bulge in the secondary structure between miR-373 and the E-Cadherin target sequence (as shown graphically in FIG. 1, panel A as a raised bulge.) Additionally, the term “miRNA” includes nucleic acids that also contain moieties other than ribonucleotide moieties, including, but not limited to, modified nucleotides, modified internucleotide linkages, non-nucleotides, deoxynucleotides and analogs of the aforementioned nucleotides. Furthermore, “pri-mRNA”, “pre-miRNA”, and “miRNA” are not limited to the method by which these molecules are produced, and may be made by recombinant means (e.g., in a cell-based system as described above or in a cell-free system using isolated enzymes) or by chemical synthesis.

The phrase “mammalian cell” refers to a cell of any mammal, including humans. The phrase refers to cells in vivo, such as, for example, in an organism or in an organ of an organism. The phrase also refers to cells in vitro, such as, for example, cells maintained in cell culture.

The term “methylation” refers to the attachment of a methyl group (—CH3) to another molecule. Typically, when DNA undergoes methylation, a methyl group is added to a cytosine bearing nucleotide, commonly at a CpG sequence, although methylation can occur at other sites as well.

The term “demethylation” refers to the removal of a methyl group (—CH3) from another molecule. Typically, when DNA undergoes demethylation, a methyl group is removed from a cytosine bearing nucleotide, commonly at a CpG sequence, although demethylation can occur at other sites as well.

The phrase “pharmaceutically acceptable carrier” refers to compositions that facilitate the introduction of miRNA into a cell and includes but is not limited to solvents or dispersants, coatings, anti-infective agents, isotonic agents, agents that mediate absorption time or release of the inventive polynucleotides and double stranded polynucleotides. Examples of “pharmaceutically acceptable carriers” include liposomes that can be neutral or cationic, can also comprise molecules such as chloroquine and 1,2-dioleoyl-sn-glycero-3-phosphatidyle-thanolamine, which can help destabilize endosomes and thereby aid in delivery of liposome contents into a cell, including a cell's nucleus. Examples of other pharmaceutically acceptable carriers include poly-L-lysine, polyalkylcyanoacrylate nanoparticles, polyethyleneimines, and any suitable PAMAM dendrimers (polyamidoamine) known in the art with various cores such as, for example, ethylenediamine cores, and various surface functional groups such as, for example, cationic and anionic functional groups, amines, ethanolamines, aminodecyl.

As used herein, “apoptosis” is a process of self-destruction in certain cells, for example, epithelial cells and erythrocytes, that are genetically programmed to have a limited life span or are damaged. Apoptosis can be induced either by a stimulus, such as irradiation or toxic drugs, by removal of a repressor agent, or by activation of pro-apoptotic genes. The cells disintegrate into membrane-bound particles that are then eliminated by phagocytosis. Also known as programmed cell death.

### Overview

The present invention provides methods and compositions for activation of a gene by introducing into the cell an miRNA wherein the miRNA molecule comprises a single ribonucleic acid strand comprising a ribonucleotide sequence complementary to a non-coding nucleic acid sequence of the gene, wherein this region of complementarity is selected so as to provide for an increase in transcription of the corresponding gene. In certain cases, the miRNA can be provided either as a double-stranded molecule or as a precursor hairpin (pre-miRNA) that can be transfected into a cell.

This invention is based in part on the surprising discovery that introduction of a miRNA molecule into a cell effects sequence specific transcriptional activation in mammalian cells.

As described in the examples in more detail, the invention is based on the discovery that miRNA targeting the E-cadherin promoter, CSDC2 promoter or Ccnb1 promoter induces high levels of mRNA expression and concomitant levels of translated polypeptide.

Mechanistically, and without wishing to be bound to theory, one model of miRNA activation of gene expression requires complementarity to targeted DNA sequences and causes changes in chromatin associated with gene activation (Li et al., (2006) Proc Natl Acad Sci USA 103, 17337-42; Janowski, et al., (2007) Nat Chem Biol 3, 166-73). Alternatively, another model for the mechanism of miRNA induced gene expression involves miRNA directly binding to complementary DNA within gene promoters to trigger gene expression. In this regard, miRNA may function like a transcription factor targeting complementary motifs in gene promoters. In yet another model, cells may be producing RNA copies of the target promoter region. Complementary miRNA would interact with the promoter RNA transcripts to result in downstream gene expression. In yet another model, the miRNA may act to recruit RNA polymerase II (RNApII) to the site, increasing the density of RNApII at the site, which increases the numbers of transcriptional complexes formed to drive increased transcription.

These observations support a fundamental role for miRNA in regulating genome structure and function and identify a therapeutic use for miRNA in targeted gene activation (e.g., increasing gene expression).

In one aspect the invention provides methods of increasing expression of a gene (i.e., gene activation) by introducing a miRNA molecule into a mammalian cell which can be accomplished by delivery of the miRNA into the cell directly (e.g. microinjection) or as a result of expression from a DNA introduced into the cell, wherein the miRNA molecule has a strand that is complementary to a region of a non-coding nucleic acid sequence of the gene, wherein the introduction results in an increase in expression of the gene. Increasing gene expression can be useful in the context of a tumor suppressor gene in, for example, inhibition of cellular proliferation, inhibition of cellular transformation and inhibition of cellular migration (e.g., as an anti-cancer agent). Increasing gene expression can be useful in the context of a pro-apoptotic gene in, for example, stimulation of the cell death program in rapidly dividing cancer cells. Increasing gene expression can be useful where long term expression of a lowly expressed gene is desired, either of an endogenous gene or of an introduced gene whose expression has decreased (e.g. gene therapy). In another aspect the invention provides compositions and pharmaceutical preparations comprising at least one miRNA molecule.

The invention will now be described in more detail.

### Compositions

As noted above the present invention provides miRNA molecules for use in performing gene activation (e.g., increase gene expression) in mammalian cells by targeting a region of non-coding nucleic acid sequence of the gene (e.g., a regulatory sequence).

As used herein the term “miRNA” herein refers to a ribonucleic acid molecule capable of facilitating gene activation and can be composed of a ribonucleic acid strand comprising a ribonucleotide sequence complementary to a non-coding nucleic acid sequence of a gene. The miRNA molecule is usually between about 10 and about 50 base pairs in length, about 12 and about 48 base pairs, about 14 and about 46 base pairs, about 16 and about 44 base pairs, about 18 and about 42 base pairs, about 20 and about 40 base pairs, about 22 and about 38 base pairs, about 24 and about 36 base pairs, about 26 and about 34 base pairs, about 28 and about 32 base pairs, normally about 10, about, 15, about 20, about 25, about 30, about 35, about 40, about 45, about 50 base pairs in length. Additionally, the term miRNA includes nucleic acids that also contain moieties other than ribonucleotide moieties, including, but not limited to, modified nucleotides, modified internucleotide linkages, non-nucleotides, deoxynucleotides and analogs of the aforementioned nucleotides.

As used herein, the terms “gene activating”, “activating a gene”, or “gene activation” are interchangeable and refer to increasing gene expression with respect to transcription as measured by transcription level, mRNA level, enzymatic activity, methylation state, chromatin state or configuration, recruitment of RNA polymerase II or other measure of its activity or state in a cell or biological system. Furthermore, “gene activating”, “activating a gene”, or “gene activation” refer to the increase of activity known to be associated with a nucleic acid sequence, such as its ability to function as a regulatory sequence, its ability to be transcribed, its ability to be translated and result in expression of a protein, regardless of the mechanism whereby such activation occurs.

miRNA compounds of the present invention can be duplexes, and can be composed of separate strands or can comprise pre-miRNAs that form short hairpin RNAs, with loops as long as, for example, about 4 to about 23 or more nucleotides, about 5 to about 22, about 6 to about 21, about 7 to about 20, about 8 to about 19, about 9 to about 18, about 10 to about 17, about 11 to about 16, about 12 to about 15, about 13 to about 14 nucleotides, RNAs with stem loop bulges, and short temporal RNAs. RNAs having loops or hairpin loops can include structures where the loops are connected to the stem by linkers such as flexible linkers. Flexible linkers can be selected of a wide variety of chemical structures, as long as they are of sufficient length and materials to enable effective intramolecular hybridization of the stem elements. Typically, the length to be spanned is at least about 10-24 atoms.

The miRNA molecules of the present invention include a region of complementarity to a non-coding region of a gene of appropriate length to provide for transcriptional activation of an adjacent coding sequence. miRNA molecules also typically include 3′ terminal nucleotides which are not complementary to the non-coding sequence. As an example, miR-373 contains 2 nucleotide overhangs at the 3′ end (FIG. 1, panel C). miRNA typically comprise more than 10 nucleotides and less than 50 nucleotides, usually more than about 12 nucleotides and less than 48 nucleotides in length, such as about 14 nucleotides to about 46 nucleotides in length, about 16 nucleotides to about 44 nucleotides in length, including about 18 nucleotides to about 42 nucleotides in length, about 20 nucleotides to about 40 nucleotides in length, about 22 nucleotides to about 38 nucleotides in length, about 24 nucleotides to about 36 nucleotides in length, about 26 nucleotides to about 34 nucleotides in length, about 28 nucleotides to about 32 nucleotides in length. In certain cases, the miRNA molecules comprise of about 14 nucleotides to about 30 nucleotides in length, such as about 15 nucleotides to about 29 nucleotides in length, about 16 nucleotides to about 28 nucleotides in length, including about 17 nucleotides to about 27 nucleotides in length, about 18 nucleotides to about 26 nucleotides in length, about 19 nucleotides to about 25 nucleotides in length, about 20 nucleotides to about 24 nucleotides in length, about 21 nucleotides to about 23 nucleotides in length, or about 22 nucleotides in length.

The miRNA molecules of the present invention typically comprises a region of complementarity greater than about 10 base pairs and less than about 50 base pairs in length. In some embodiments, the miRNA molecules comprise a duplex region of between about 12 base pairs to about 48 base pairs in length, such as about 14 base pairs to about 46 base pairs in length, about 16 base pairs to about 44 base pairs in length, including about 18 base pairs to about 42 base pairs in length, about 20 base pairs to about 40 base pairs in length, about 22 base pairs to about 38 base pairs in length, about 24 base pairs to about 36 base pairs in length, about 26 base pairs to about 34 base pairs in length, about 28 base pairs to about 32 base pairs in length. In representative embodiments, the miRNA molecules comprise of a duplex region between about 15 base pairs to about 30 base pairs in length, such as about 16 base pairs to about 29 base pairs in length, about 17 base pairs to about 28 base pairs in length, including about 18 base pairs to about 27 base pairs in length, about 19 base pairs to about 26 base pairs in length, about 20 base pairs to about 25 base pairs in length, about 21 base pairs to about 24 base pairs in length, about 22 base pairs to about 23 base pairs in length. For example, a miR-373 which induces expression of a human E-cadherin gene, comprises a 20 nucleotide region of complementarity to a non-coding region of E-cadherin and has 23 nucleotides of total sequence.

In representative embodiments, the miRNA molecules comprise a strand that is complementary to a portion of a non-coding nucleic acid sequence or a gene, e.g., a regulatory sequence, such as a promoter. In some embodiments, the strand is 100% complementary to the non-coding nucleic acid sequence of the gene, including about 99% complementary, 98% complementary, 97% complementary, 96% complementary, 95% complementary, 94% complementary, 93% complementary, 92% complementary, 91% complementary, 90% complementary, 85% complementary, 80% complementary, 75% complementary, 70% complementary to the non-coding nucleic acid sequence of the gene.

As described in greater detail above, the miRNA molecules of the present invention comprises a strand that is complementary to a portion of a non-coding nucleic acid sequence or a gene, e.g., a regulatory sequence, such as a promoter. When designing the complementary strand of the miRNA molecules of the invention (e.g., the strand of the miRNA molecule that is complementary to a portion of a non-coding nucleic acid sequence or a gene), the sequence is selected so as to avoid complementarity to any CpG island regions. By “CpG island region” is meant any region of the nucleic acid that is rich in the dinucleotide “CG” (Cytosine-Guanine). Methylation of the cytosine in the dinucleotide is maintained through cell divisions and affects the degree of transcription of the nearby genes by silencing gene expression and is important in developmental regulation of gene expression. In certain cases, avoiding CpG islands serves to avoid methylation of the cytosine residue of CpG island regions and thereby silencing expression of the nearby gene. This is shown, for example, graphically in FIG. 1, panel A.

In addition, when designing the complementary strand of the miRNA molecules of the invention (e.g., the strand of the miRNA molecule that is complementary to a portion of a non-coding nucleic acid sequence of a gene), the sequence is selected so as to avoid complementarity to any GC-rich regions. By “GC-rich” is meant any region of the nucleic acid that includes a greater number of guanine and cytosine base pairs compared to thymine and adenine base pairs as compared to the average number of guanine and cytosine residues in the rest of the genome in which the nucleic acid is present.

A CpG island region or a GC-rich region can be determined, for example, by using a prediction protocol, such as for example the CpGPlot/CpGReport/lsochore protocol available on the world wide web at ebi.ac.uk/emboss/cpgplot/ or the MethPrimer protocol available on the world wide web at urogene.org/methprimer/index1.html. Scanning the non-coding promoter region of the E-cadherin and CSDC2 genes can be performed, for example, by using the software RegRNA, which can be found on the world wide web at regrna.mbc.nctu.edu.tw or by contacting the Department of Biological Science and Technology, Institute of Bioinformatics, National Chiao Tung University, Taiwan.

In addition, the miRNA molecules of the subject invention will typically be designed in order to avoid a non-coding nucleic acid sequence of a gene comprising a GC content greater than about 50% or less than about 30%. In certain embodiments, the miRNA molecules of the subject invention will be designed in order to comprise a GC content greater than about 30% or less than about 50%, including a GC content of about 32%, a GC content of about 34%, a GC content of about 36%, a GC content of about 38%, a GC content of about 40%, a GC content of about 42%, a GC content of about 44%, a GC content of about 46%, a GC content of about 48%, a GC content of about 50%.

Likewise, the miRNA molecules of the subject invention will typically be designed in order comprise an AT content greater than about 50% to less than about 80%. In certain embodiments, the miRNA molecules of the subject invention will be designed in order to comprise an AT content of about 52%, an AT content of about 54%, an AT content of about 56%, an AT content of about 58%, an AT content of about 60%, an AT content of about 62%, an AT content of about 64%, an AT content of about 66%, an AT content of about 68%, an AT content of about 70%, an AT content of about 72%, an AT content of about 74%, an AT content of about 76%, an AT content of about 78%, an AT content of about 80%.

Moreover, the miRNA molecules of the subject invention will typically be designed in order to avoid a non-coding nucleic acid sequence of a gene comprising single nucleotide polymorphism (SNP) sites. Without being held to theory, avoiding GC rich regions, repeats, and non-complex sequences serves to avoid “slippage” of the miRNA when duplexed to the target sequence (e.g., a GC-rich sequence may cause the miRNA to anneal to the target in a manner that adversely affects the overall desired region of complementarity with the target).

The miRNA molecules of the present invention include a region of complementarity to non-coding target nucleic acid sequence. A non-coding target nucleic acid sequence refers to a nucleic acid sequence of interest that is not contained within an exon or is a regulatory sequence. In general, such a non-coding target sequence is a nucleic acid sequence approximately 2 kb upstream from the transcriptional start site of the target gene, including up to about 1.9 kb, about 1.8 kb, about 1.7 kb, about 1.6 kb, about 1.5 kb, about 1.4 kb, about 1.3 kb, about 1.2 kb, about 1.1 kb, about 1 kb, about 950 bp, about 900 bp, about 850 bp, about 800 bp, about 750 bp, about 700 bp, about 650 bp, about 600 bp, about 550 bp, about 500 bp, about 450 bp, about 400 bp, about 350 bp, about 300 bp, about 250 bp, about 200 bp, about 150 bp, about 100 bp, about 50 bp, and the like.

In certain embodiments, the non-coding target nucleic acid sequence may include any enhancer sequence within about a 5 kb region upstream of the transcriptional start site of the target gene, including about 4.5 kb, about 4 kb, about 3.5 kb, about 3 kb, about 2.5 kb, about 2 kb, about 1.5 kb, and the like. In other embodiments, the non-coding target nucleic acid sequence may include the first intron sequence downstream of the transcriptional start site of the target gene.

The strand(s) of the miRNA molecule may have terminal (5′ or 3′) overhang regions of any length that are non-base pairing nucleotide resulting from one strand extending beyond the other strand within a doubled stranded polynucleotide. In addition, the overhang regions are also not complementary (a region of non-complementarity) to the non-coding sequence of the gene. If they have overhang regions, these regions in representative embodiments are 8 nucleotides or fewer in length, 7 nucleotides or fewer in length, 6 nucleotides or fewer in length, including about 5 nucleotides, about 4 nucleotides, such as about 3 nucleotides or fewer in length, including about two nucleotides in length, and about one nucleotide in length. In such embodiments, the regions are further described by the following formula:

3′-N₍₁₊ₙ₎-miRNA-5′, or

5′-N₍₁₊ₙ₎-miRNA-3′

wherein N is any nucleotide, including naturally-occurring or non-naturally-occurring, genetically encodable or non-genetically encodable, residue and n is any integer from 0 to 7.

The nucleotides of the miRNA, or at least one strand of a duplex miRNA, may be modified so as to provide a desired characteristic.

The miRNA may be synthesized by any method that is now known or that comes to be known for synthesizing miRNA molecules and that from reading this disclosure, one skilled in the art would conclude would be useful in connection with the present invention. For example, one may use methods of chemical synthesis such as methods that employ Dharmacon, Inc.'s proprietary ACE® technology. Alternatively, one could also use template dependant synthesis methods. Synthesis may be carried out using modified or non-modified, natural or non-natural bases as disclosed herein. Moreover, synthesis may be carried out with or without modified or non-modified nucleic acid backbone as disclosed herein.

In addition, the miRNA molecules may be synthesized in a host cell by any method that is now known or that comes to be known for synthesizing miRNA molecules in a host cell. In addition, the miRNA may be synthesized as a pre-miRNA and further processed by the host cell, thus providing a miRNA. For example, miRNA or pre-miRNA molecules can be expressed from recombinant circular or linear DNA vector using any suitable promoter. Suitable promoters for expressing miRNA or pre-mRNA molecules of the invention from a vector include, for example, the U6 or H1 RNA pol III promoter sequences and the cytomegalovirus promoter. Selection of other suitable promoters is within the skill in the art. Suitable vectors for use with the subject invention include those described in U.S. Pat. No. 5,624,803, the disclosure of which is incorporated herein in its entirely. The recombinant plasmids of the invention can also comprise inducible or regulatable promoters for expression of the miRNA molecule in a particular tissue or in a particular intracellular environment.

Selection of vectors suitable for expressing miRNA of the invention, methods for inserting nucleic acid sequences for expressing either the miRNA or the pre-miRNA into the vector, and methods of delivering the recombinant vector to the cells of interest are within the skill in the art. See, for example Tuschl et al., (2002), Nat. Biotechnol, 20: 446-448; Brummelkamp et al., (2002), Science 296: 550-553; Miyagishi et al., (2002), Nat. Biotechnol. 20: 497-500; Paddison et al. (2002), Genes Dev. 16: 948-958; Lee et al., (2002), Nat. Biotechnol. 20: 500-505; and Paul et al., (2002), Nat. Biotechnol. 20: 505-508, the entire disclosures of which are herein incorporated by reference. Other methods for delivery and intracellular expression suitable for use in the invention are described in, for example, U.S. Patent Application Publication Nos. 20040005593, 20050048647, 20050060771, the entire disclosures of which are herein incorporated by reference.

### Methods

The present invention provides methods of increasing gene expression comprising introducing a miRNA molecule into a mammalian cell, wherein the miRNA molecule has a strand that is complementary to a region of a non-coding nucleic acid sequence of the gene, wherein the introduction results in an increase in expression of the gene. In general, “increasing gene expression” refers to an increase in the gene's ability to be transcribed, its ability to be translated and result in expression of a protein, regardless of the mechanism whereby such activation occurs.

In certain cases, the methods of the present invention are carried out by contacting a cell with a miRNA molecule, wherein the miRNA molecule comprises a ribonucleic acid strand comprising a ribonucleotide sequence complementary to a non-coding nucleic acid sequence of a gene, wherein the introduction results in an increase in expression of the gene.

In certain cases, an increase in gene expression results in a detectable increase above control (i.e. in the absence of the miRNA molecule) or more in transcription associated with a nucleic acid sequence. In some embodiments, the increase in gene expression results in at least about a 2.0 fold increase or more, at least about a 2.5-fold increase or more, at least about a 3-fold increase or more, at least about a 3.5-fold increase or more, at least about a 4-fold increase or more, at least about a 4.5-fold increase or more, at least about a 5-fold increase or more, at least about a 5.5-fold increase or more, at least about a 6-fold increase or more, at least about a 6.5-fold increase or more, at least about a 7-fold increase or more, at least about a 7.5-fold increase or more at least about a 8-fold increase or more, and up to about 10-fold increase or more, including about 15-fold increase or more, about 20-fold increase or more, such as 25-fold increase or more. An increase in gene expression or activity can be measured by any of a variety of methods well known in the art. Suitable methods of examining gene expression or activity include measuring nucleic acid transcription level, mRNA level, level of the translated polypeptide, enzymatic activity, methylation state, chromatin state or configuration, or other measure of nucleic acid activity or state in a cell or biological system. After introduction of a miRNA molecule into the cell, the introduction may result in an increase in the density of RNA polymerase II (RNApII) in the promoter region targeted by the miRNA, and is an alternative method of examining gene expression or activity.

Because the ability of the miRNA of the present invention to retain functionality either as a miRNA or as a pre-miRNA, it is not limited to the cell type, or the species into which it is introduced, the present invention is applicable across a broad range of mammals, including but not limited to humans. The present invention is particularly advantageous for use in mammals such as cattle, horse, goats, pigs, sheep, canines, rodents such as hamsters, mice, and rats, and primates such as, for example, gorillas, chimpanzees, and humans. Transgenic mammals may also be used, e.g. mammals that have a chimeric gene sequence. Methods of making transgenic animals are well known in the art; see, for example, U.S. Pat. No. 5,614,396.

The present invention may be used advantageously with diverse cell types including those of the germ cell line, as well as somatic cells. The cells may be stem cells or differentiated cells. For example, the cell types may be embryonic cells, oocytes sperm cells, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes and cells of the endocrine or exocrine glands.

The present invention is applicable for use for activation (e.g., increasing expression) of a broad range of genes, including but not limited to the genes of a human genome, such as those implicated in diseases such as diabetes, Alzheimer's and cancer, as well as all genes in the genomes of the aforementioned organisms. The miRNAs of the invention may be useful in activating genes for the production of drugs (e.g. protein growth hormone, EPO or insulin) or the production of commercially desirable proteins (e.g. serum proteins, lactase etc.). The present invention may be applicable in the field of cancer, for example, inducing the expression of tumor suppressor genes. In a similar aspect, the miRNAs of the invention may be useful in activating pro-apoptotic genes (e.g Bax), when it is desirable to remove a specific population of cells (e.g. cancer cells) from a population by programmed cell death. Furthermore, the compositions and methods of the present invention may also be used to target a recombinant gene, such as a gene introduced on a nucleic acid or viral vector.

The compositions and methods of the present invention may be administered to a cell or applied by any method that is now known or that comes to be known and that from reading this disclosure, one skilled in the art would conclude would be useful with the present invention. For example, the miRNA may be passively delivered to cells. Passive uptake of modified miRNA can be modulated, for example, by the presence of a conjugate such as a polyethylene glycol moiety or a cholesterol moiety at the 5′ terminal of the sense strand and/or, in appropriate circumstances, a pharmaceutically acceptable carrier.

The miRNA may be delivered to a cell by any method that is now known or that comes to be known and that from reading this disclosure, persons skilled in the art would determine would be useful in connection with the present invention in enabling miRNA to cross the cellular membrane and/or the nuclear membrane. These methods include, but are not limited to, any manner of transfection, such as for example transfection employing DEAE-Dextran, calcium phosphate, cationic lipids/liposomes, micelles, manipulation of pressure, microinjection, electroporation, immunoporation, use of vectors such as viruses (e.g., RNA virus), plasmids, cell fusions, and coupling of the polynucleotides to specific conjugates or ligands such as antibodies, antigens, or receptors, passive introduction, adding moieties to the miRNA that facilitate its uptake, and the like.

The miRNAs of the present invention may be used in a diverse set of applications, including but not limited to basic research, drug discovery and development, diagnostics and therapeutics. For example, the present invention may be used to validate whether a gene product is a target for drug discovery or development. In this application, a target nucleic acid sequence of interest is identified for activation (e.g., increasing expression). For example, a cell is contacted with a miRNA and the extent of any increased activity, such as, for example, transcription or translation, of the gene is then assessed, along with the effect of such increased activity, and a determination is made that if activity is increased, then the nucleic acid sequence of interest is a target for drug discovery or development. In this manner, phenotypically desirable effects can be associated with miRNA activation of particular target nucleic acids of interest, and in appropriate cases toxicity and pharmacokinetic studies can be undertaken and therapeutic preparations developed.

The present invention may also be used in applications that induce transient or permanent states of disease or disorder in an organism by, for example, increasing the activity (e.g., by increasing transcription or translation) of a target nucleic acid of interest believed to be a cause or factor in the disease or disorder of interest in order to provide an animal model of a disease or disorder. Increased activity of the target nucleic acid of interest may render the disease or disorder worse, or tend to ameliorate or to cure the disease or disorder of interest, as the case may be. Likewise, increased activity of the target nucleic acid of interest may cause the disease or disorder, render it worse, or tend to ameliorate or cure it, as the case may be.

Target nucleic acids of interest can comprise genomic or chromosomal nucleic acids or extrachromosomal nucleic acids, such as viral nucleic acids. Target nucleic acids of interest can include all manner of nucleic acids, such as, for example, non-coding DNA, regulatory DNA, repetitive DNA, reverse repeats, centromeric DNA, DNA in euchromatin regions, DNA in heterochromatin regions, promoter sequences, enhancer sequences, introns sequences, exon sequences, and the like.

Still further, the present invention may be used in applications, such as diagnostics, prophylactics, and therapeutics. For these applications, an organism suspected of having a disease or disorder that is amenable to modulation by manipulation of a particular target nucleic acid of interest is treated by administering miRNA. Results of the miRNA treatment may be ameliorative, palliative, prophylactic, and/or diagnostic of a particular disease or disorder. In representative embodiments, the miRNA is administered in a pharmaceutically acceptable manner with a pharmaceutically acceptable carrier with or without a diluent.

In some embodiments increasing expression of tumor suppressor genes is desirable. As such, agents that act to increase gene activity in such genes are useful in the treatment of a cellular proliferative disease, e.g., any condition, disorder or disease, or symptom of such condition, disorder, or disease that results from the uncontrolled proliferation of cells, e.g., cancer. Cancer is an example of a condition that is treatable using the compounds of the invention. Use of the miRNA of the invention in combination with a second compound for use in treatment of a cellular proliferative disease is of particular interest. Exemplary cancers suitable for treatment with the subject methods include colorectal cancer, non-small cell lung cancer, small cell lung cancer, ovarian cancer, breast cancer, head and neck cancer, renal cell carcinoma, and the like.

Exemplary tumor suppressor genes include, but are not limited to, p53, p21, BRCA1, BRCA2, APC, RB1, CDKN2A, DCC, DPC4 (SMAD4), MADR2/JV18 (SMAD2), MEN1, MTS1, NF1, NF2, PTEN, VHL, WRN, and WT1. Other genes of interest include, but are not limited to, the nitric oxide synthase (NOS) genes, including NOS1 (nNOS) and NOS3 (eNOS), e-cadherin, growth factors, such as vascular endothelial growth factor (VEGF), neuronal growth factor (NGF), epidermal growth factor (EGF), fibroblast growth factors (FGFs) and the like.

Additional exemplary genes of interest include CSDC2/PIPPIN (Cold Shock Domain Containing C2), NPM1 (nucleophosmin 1), NUPL2 (nucleoporin like 2), BFAR (bifunctional apoptosis regulator), IRAK3 (interleukin-1 receptor-associated kinase 3), GAA (Lysosomal alpha-glucosidase precursor), NSUN7 (NOL1/NOP1/Sun domain family member 7), RAGE (Renal tumor antigen 1), FOXP2 (Forkhead Box P2), RBKS (Ribokinase), ACOT6 (acyl-CoA thioesterase 6), SIRPD (Signal Regulatory Protein Delta), and CCNB1 (cyclin B).

In some embodiments increasing expression of pro-apoptotic genes is desirable. As such, agents that act to increase gene activity in such genes are useful in the treatment of a cellular proliferative disease, e.g., any condition, disorder or disease, or symptom of such condition, disorder, or disease that results from the uncontrolled proliferation of cells, e.g., cancer. An increase in apoptosis increases the amount of cell death, reducing the number of uncontrolled proliferating cells found in the cancer. Cancer is an example of a condition that is treatable using the compounds of the invention. Use of the miRNA of the invention in combination with a second compound for use in treatment of a cellular proliferative disease is of particular interest. Exemplary cancers suitable for treatment with the subject methods include colorectal cancer, non-small cell lung cancer, small cell lung cancer, ovarian cancer, breast cancer, head and neck cancer, renal cell carcinoma, and the like. In certain cases, pro-apoptotic genes include, but are not limited to; Bax, SMAC, Bak, Diva, Bcl-Xs, Bik, Bim, Bad, Bid, Noxa, BID, PUMA and Egl-1.

Subjects suitable for treatment with a method of the present invention involving miRNAs include individuals having a cellular proliferative disease, such as a neoplastic disease (e.g., cancer). Cellular proliferative disease is characterized by the undesired propagation of cells, including, but not limited to, neoplastic disease conditions, e.g., cancer. Examples of cellular proliferative disease include, but are not limited to, abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying, for example, rheumatoid arthritis, psoriasis, diabetic retinopathy, other ocular angiogenic diseases such as retinopathy of prematurity (retrolental fibroplastic), macular degeneration, corneal graft rejection, neuroscular glaucoma and Oster Webber syndrome, psoriasis, restinosis, fungal, parasitic and viral infections such as cytomegaloviral infections. Subjects to be treated according to the methods of the invention include any individual having any of the above-mentioned disorders.

The invention should not be construed to be limited solely to the treatment of patients having a cellular proliferative disease. Rather, the invention should be construed to include the treatment of patients having conditions or disease associated with decreased expression of particular genes that would benefit from the methods of the subject invention.

Such subjects may be tested in order to assay the activity and efficacy of the subject miRNA. A significant improvement in one or more of parameters is indicative of efficacy. It is well within the skill of the ordinary healthcare worker (e.g., clinician) to adjust dosage regimen and dose amounts to provide for optimal benefit to the patient according to a variety of factors (e.g., patient-dependent factors such as the severity of the disease and the like, the compound administered, and the like).

Pharmaceutical Preparations Containing Compounds of the Invention

Also provided by the invention are pharmaceutical preparations of the subject miRNA compounds described above. The subject miRNA compounds can be incorporated into a variety of formulations for therapeutic administration by a variety of routes. More particularly, the compounds of the present invention can be formulated into pharmaceutical compositions by combination with appropriate, pharmaceutically acceptable carriers, diluents, excipients and/or adjuvants, and may be formulated into preparations in solid, semi-solid, liquid or gaseous forms, such as tablets, capsules, powders, granules, ointments, solutions, suppositories, injections, inhalants and aerosols, in a sterile vial or in a syringe. Where the formulation is for transdermal administration, the compounds are preferably formulated either without detectable DMSO or with a carrier in addition to DMSO. The formulations may be designed for administration to subjects or patients in need thereof via a number of different routes, including oral, buccal, rectal, parenteral, intraperitoneal, intradermal, intratracheal, etc. The administration can be systemic or localized delivery of the formulation to a site in need of treatment, e.g., localized delivery to a tumor.

Pharmaceutically acceptable excipients usable with the invention, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

Dosage Forms of Compounds of the Invention

In pharmaceutical dosage forms, the subject miRNA compounds of the invention may be administered in the form of their pharmaceutically acceptable salts, or they may also be used alone or in appropriate association, as well as in combination, with other pharmaceutically active compounds. The following methods and excipients are merely exemplary and are in no way limiting.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes, such as intrapulmonary or intranasal delivery.

Conventional and pharmaceutically acceptable routes of administration include intranasal, intrapulmonary intramuscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses.

For oral preparations, the subject miRNA compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Parenteral routes of administration other than inhalation administration include, but are not necessarily limited to, topical, transdermal, subcutaneous, intramuscular, intraorbital, intracapsular, intraspinal, intrasternal, intravenous routes, i.e., any route of administration other than through the alimentary canal, and local injection, with intra or peritumoral injection being of interest, especially where a tumor is a solid or semi-solid tumor (e.g., Hodgkins lymphoma, non-Hodgkins lymphoma, and the like). Local injection into a tissue defining a biological compartment (e.g., prostate, ovary, regions of the heart (e.g., pericardial space defined by the pericardial sac), intrathecal space, synovial space, and the like) is also of interest. Parenteral administration can be carried to effect systemic or local delivery of the agent. Where systemic delivery is desired, administration typically involves invasive or systemically absorbed topical or mucosal administration of pharmaceutical preparations.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

The subject miRNA compounds of the invention can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol, collagen, cholesterol; and if desired, with conventional additives such as solubilizers, isotonic agents, suspending agents, emulsifying agents, stabilizers and preservatives.

The miRNA compounds of the invention can also be delivered to the subject by enteral administration. Enteral routes of administration include, but are not necessarily limited to, oral and rectal (e.g., using a suppository) delivery.

Furthermore, the subject miRNA compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Dosages of the Compounds of the Invention

Depending on the subject and condition being treated and on the administration route, the subject miRNA compounds may be administered in dosages of, for example, 0.1 μg to 100 mg/kg body weight per day. In certain embodiments, the therapeutic administration is repeated until a desired effect is achieved. Similarly the mode of administration can have a large effect on dosage. Thus, for example, oral dosages may be about ten times the injection dose. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill in the art will readily appreciate that dose levels can vary as a function of the specific compound, the severity of the symptoms and the susceptibility of the subject to side effects. Dosages for a given compound are readily determinable by those of skill in the art by a variety of means.

Although the dosage used will vary depending on the clinical goals to be achieved, a suitable dosage range is one which provides up to about 1 μg to about 1,000 μg or about 10,000 μg of subject composition to reduce a symptom in a subject animal.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the invention. Similarly, unit dosage forms for injection or intravenous administration may comprise the compound (5) in a composition as a solution in sterile water, normal saline or another pharmaceutically acceptable carrier.

Combination Therapy Using the Compounds of the Invention

For use in the subject methods, the subject compounds may be formulated with or otherwise administered in combination with other pharmaceutically active agents, including other agents that activate or suppress a biochemical activity, such as a chemotherapeutic agent. The subject compounds may be used to provide an increase in the effectiveness of another chemical, such as a pharmaceutical, or a decrease in the amount of another chemical, such as a pharmaceutical that is necessary to produce the desired biological effect.

Examples of chemotherapeutic agents for use in combination therapy include, but are not limited to, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES).

Furthermore, the miRNA compounds of the present invention may also be used in combination therapy with other miRNA molecules. In such embodiments, the miRNA molecules may be administered to increase activation of a first gene (activating RNA-RNAa) and a second RNAi (inhibitory RNA-RNAi) molecule designed to reduce gene expression may be administered to silence expression of a second gene. For example, the miRNA molecules of the invention may be administered to increase activation of a tumor suppressor gene or a pro-apoptotic gene, and the RNAi molecule may be administered to silence expression of an oncogene.

The compounds described herein for use in combination therapy with the compounds of the present invention may be administered by the same route of administration (e.g. intrapulmonary, oral, enteral, etc.) that the compounds are administered. In the alternative, the compounds for use in combination therapy with the compounds of the present invention may be administered by a different route of administration that the compounds are administered.

### Kits

Kits with unit doses of the subject compounds, usually in oral or injectable doses, are provided. In such kits, in addition to the containers containing the unit doses will be an informational package insert describing the use and attendant benefits of the drugs in treating pathological condition of interest. Representative compounds and unit doses are those described herein above.

In one embodiment, the kit comprises a miRNA formulation in a sterile vial or in a syringe, which formulation can be suitable for injection in a mammal, particularly a human.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### miRNA Specifically Targets the E-Cadherin Promoter

The RegRNA program was used to scan promoter regions in silico. The software is designed to scan input RNA sequences for regulatory motifs and miRNA target sites. However, by manipulating a promoter sequence, the software may identify putative sites complementary to known miRNAs in DNA strands. Scanning analysis revealed a sequence located at position minus(−) 645 nucleotides in the 5′ direction relative to the transcription start site in the E-cadherin promoter highly complementary to miR-373 (FIG. 1, panel A and FIG. 1, panel B). The native duplex of miR-373 was synthesized, as well as another dsRNA molecule (dsEcad-640) that was fully (100%) complementary to the putative miR-373 target site (FIG. 1, panel C). A control dsRNA (dsControl) was synthesized that lacked significant homology to all known human sequences. Each dsRNA duplex was transfected into PC-3 prostate cancer cells and E-cadherin expression was analyzed 72 hours later.

PC-3, LNCaP, and HCT-116 cells were maintained in RPMI 1640 medium supplemented with 10% FBS, 2 mM L-glutamine, penicillin (100 U/ml) and streptomycin (100 μg/ml) in a humidified atmosphere of 5% CO₂ at 37° C. Tera-1 cells were cultured in McCoy's 5A media supplemented with 10% FBS, 2 mM L-glutamine, penicillin and streptomycin. The day before dsRNA transfection, cells were plated in growth medium without antibiotics at a density of about 50%-60%. Transfection of dsRNA was carried out by using Lipofectamine 2000 (Invitrogen, Carlsbad Calif.) according to the manufacture's protocol.

Preparation of miRNAs, native miR-373, mismatched derivatives of miR-373 such as miR-373-5MM and miR-373-3MM, promoter-specific dsRNAs such as dsEcad-640, dsEcad-640, dsEcad-215, and dsCSDC2-670, and the non-specific control dsRNA (dsControl) were synthesized by Invitrogen (Carlsbad, Calif.). All dsRNA/miRNA duplexes possessed 2-nucleotide 3′ overhangs. The miR-373 precursor (pre-miR-373) was synthesized by Dharmacon (Lafayette, Colo.). Pre-miR® Negative Control #1 (pre-miR-Con) was purchased from Ambion (Austin, Tex.) and served as a non-specific premature miRNA control. Anti-miR-miR-373 inhibitory oligonucleotide (anti-miR-373) and Anti-miR® Negative Control #1 (anti-miR-Con) were also acquired from Ambion. Exemplary sequences are listed below.

| | Sequence (5′-3′) | |
|---|---|---|
| miR-373 S | 5′ACUCAAAAUGGGGGUGCUUUCC 3′ | SEQ ID NO: 2 |
| miR-373 SA | 5′GAAGUGCUUCGAUUUUGGGGUGU 3′ | SEQ ID NO: 3 |
| dsEcad-640 S | 5′CCUGAAAUCCUAGCACUUU[dT][dT] 3′ | SEQ ID NO: 4 |
| dsEcad-640 AS | 5′AAAGUGCUAGGCUUUCAGG[dT][dT] 3′ | SEQ ID NO: 5 |
| miR-373-5MM S | 5′ACUCAAAAUGGGGGCUAGAUCC 3′ | SEQ ID NO: 6 |
| miR-373-5MM AS | 5′GUCUAGCUUCGAUUUUGGGGUGU 3′ | SEQ ID NO: 7 |
| miR-373-3MM S | 5′GGUGAAAAUGGGGGCGCUUUCC 3′ | SEQ ID NO: 8 |
| miR-373-3MM AS | 5′GAAGUGCUUCGAUUUUGCACCGU 3′ | SEQ ID NO: 9 |
| dsCSDC2-670 S | 5′GUUCACCUGUGCACCUUCA[dT][dT] 3′ | SEQ ID NO: 10 |
| dsCSDC2-670 AS | 5′UGAAGGUGCACAGGUGAAC[dT][dT] 3′ | SEQ ID NO: 11 |
| dsEcad-215 S | 5′AACCGCGCAGGUCCCAUAA[dT][dT] 3′ | SEQ ID NO: 12 |
| dsEcad-215 AS | 5'UUAUGGGACCUGCACGGUU[dT][dT] 3′ | SEQ ID NO: 13 |
| pre-miR-373 | 5′ACUCAAAAUGGGGGCGCUUUCCUUUUUGUCUGUACUGGGAA | SEQ ID NO: 14 |
| | GUGCUUCGAUUUUGGGGUGU 3′ | |
| RT-PCR Primers | Sequence (5′-3′) | |
| E-cadherin S | 5′CCTGGGACTCCACCTACAGA 3′ | SEQ ID NO: 15 |
| E-cadherin AS | 5′GGATGACACAGCGTGAGAGA 3′ | SEQ ID NO: 16 |
| CSDC2 S | 5′GTTCAAGGGCGTCTGTAAGC 3′ | SEQ ID NO: 17 |
| CSDC2 AS | 5′AGCTGAGTGAGCACCACCTC 3′ | SEQ ID NO: 18 |
| GAPDH S | 5′TCCCATCACCATCTTCCA 3′ | SEQ ID NO: 19 |
| GAPDH AS | 5′CATCACGCCACAGTTTCC 3′ | SEQ ID NO: 20 |
| PMO | Sequence (5′-3′) | |
| Dicer-PMO | 5′AGCAGGGCTTTTCATTCATCCAGTG 3′ | SEQ ID NO: 21 |
| ChIP Primers | Sequence (5′-3′) | |
| E-cadherin S | 5′ATAACCCACCTAGACCCTAGCAA 3′ | SEQ ID NO: 22 |
| E-cadherin AS | 5′CTCACAGGTGCTTTGCAGTTC 3′ | SEQ ID NO: 23 |
| CSDC2 S | 5′AAGCAGGGACTACAAATTCTCATC 3′ | SEQ ID NO: 24 |
| CSDC2 AS | 5′CTCTGTCTCTCTCTGGCTCGTG 3′ | SEQ ID NO: 25 |
| GAPDH S | 5′TACTAGCGGTTTTACGGGCGCACGT 3′ | SEQ ID NO: 26 |
| GAPDH AS | 5′TCGAACAGGAGGAGCAGAGAGCGAA 3′ | SEQ ID NO: 27 |

Analysis of E-cadherin mRNA expression revealed a significant increase in E-cadherin levels following miR-373 and dsEcad-640 transfections (FIG. 1, panel D). In comparison to mock transfections, both miR-373 and dsEcad-640 increased E-cadherin mRNA levels by about 7-fold (FIG. 1, panel E). The combination transfection of miR-373 and dsEcad-640 did not further increase E-cadherin expression shows that both dsRNA molecules are, in fact, targeting the same site (FIG. 1, panel D and FIG. 1, panel E). The level of E-cadherin polypeptide corresponded to the induction of E-cadherin mRNA expression as confirmed by immunoblot analysis. As shown in FIG. 1, panel F, elevated levels of E-cadherin polypeptide strongly correlate to the increase in E-cadherin mRNA expression. Thus, these results show that contrary to previously held beliefs about miRNAs only suppressing gene expression, a miRNA targeting a non-coding region of a gene (e.g. a promoter) may induce expression.

### Example 2

### Pre-miRNA Biogenesis is Required for E-Cadherin Induction

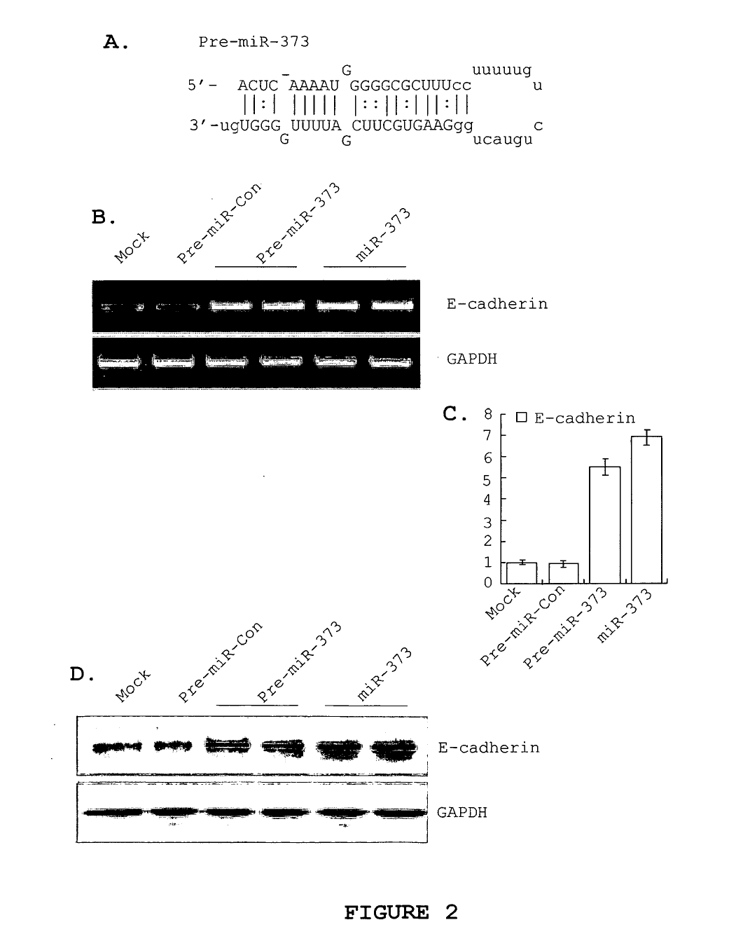

As discussed above, miRNAs are processed from precursor miRNA (pre-miRNA) hairpins by the RNase III enzyme Dicer (Ketting et al., (2001) Genes Dev 15, 2654-9). To determine if the precursor to miR-373 (pre-miR-373) could facilitate E-cadherin induction, the RNA sequence of pre-miR-373 (FIG. 2, panel A) was transfected into PC-3 cells using the methods described in Example 1. As a control, a non-specific pre-miRNA (pre-miR-Con) was tranfected. Seventy-two hours after transfection, there was a significant induction in E-cadherin mRNA expression in pre-MiR-373 cells similar to the E-cadherin induction seen in miR-373 transfected cells (FIG. 2, panel B). Compared to mock transfections, pre-miR-373 and miR-373 increased E-cadherin mRNA levels by about 5.5- and about 7-fold, respectively (FIG. 2, panel C).

Immunoblot analysis also revealed a robust increase in E-cadherin polypeptide levels (FIG. 2, panel D). This indicates that the increases in mRNA expression induced by miR-373 correlate with increases in protein level. Overall, no changes in E-cadherin expression were detected in cells transfected with mock, dsControl, or pre-miR-Con controls.

Total RNA, including miRNA, was extracted using the miRNeasy Mini Kit (Qiagen, Velencia, Calif.) according to the manufacturer's protocol. Reverse transcription reactions containing 200 ng of total RNA were performed using the TaqMan® MicroRNA Reverse Transcription Kit (Applied Biosystems, Foster City, Calif.) in conjunction with miR-373-specific primers. One microgram of total RNA was also reverse transcribed using oligo(dT) primers for analysis of GAPDH expression. In order to quantify miR-373 expression, real-time PCR was performed using the TaqMan® miRNA assay kit (Applied Biosystems). Amplification of GAPDH served as an endogenous control used to normalize miR-373 expression data. Each sample was analyzed in quadruplicate. Note that the miR-373-specific primer utilized in the reverse transcription reaction recognizes both miR-373 and pre-miR-373. Therefore, results are a quantitative measurement of the combined cellular levels of miR-373 and pre-miR-373.

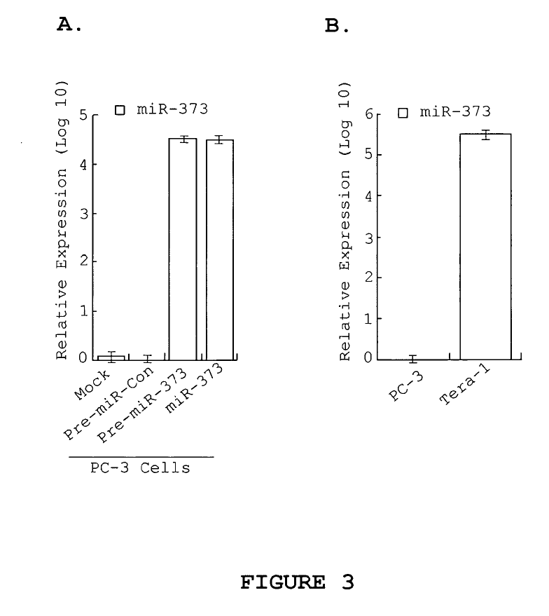

The cellular levels of miR-373 were analyzed by real-time PCR in order to quantify miR-373 in transfected cells. The level of miR-373 detected in miR-373 and pre-miR-373 transfected cells was about 30,000-times greater in transfected cells than control samples as shown in FIG. 3, panel A. This indicates there was efficient conversion of pre-miR-373 by Dicer into miR-373. The level of miR-373 found in the transfected cells is similar to that of a cell line that produces wild type miR-373 endogenously. FIG. 3, panel B is a graphical representation of the levels of miR-373 in PC-3 cells which do not express miR-373, and Tera-1 cells which express wild type miR-373. The approximately equal levels of miR-373, pre-miR-373 and miR-373 in Tera-1 cells indicates that miR-373 approaches similar cellular levels following transfection by pre-miR373 and correlates with the induction of E-cadherin in PC-3 cells.

As the processing of pre-miR373 to miR-373 most likely requires an active Dicer polypeptide, an antisense phosphorodiamidate morpholino oligonucleotide (PMO) targeting the translation initiation site in the Dicer mRNA was synthesized. The PMO serves to obstruct protein synthesis and allows for the knockdown of Dicer function without utilizing any endogenous enzymatic machinery that may also be required for miRNA-induced gene expression.

The antisense PMO molecule (Dicer-PMO) was designed and synthesized against the translation initiation site in the Dicer mRNA (Gene Tools, LLC, Corvallis, Oreg.). A standard control oligo was also purchased from Gene Tools, and served as a non-specific control PMO (Con-PMO). To block Dicer protein synthesis, semi-confluent PC-3 cells were transfected as in Example 1 with 15 μM Dicer-PMO using the Endo-Porter delivery system (Gene Tools, LLC). Control treatments were transfected in absence of PMO molecules. The following day, treated cells were reseeded at about 50%-60% confluence and immediately transfected with or without miRNA using Lipofectamine 2000 (Invitrogen, Carlsbad Calif.). All treatments proceeded for 72 hours following miRNA transfection.

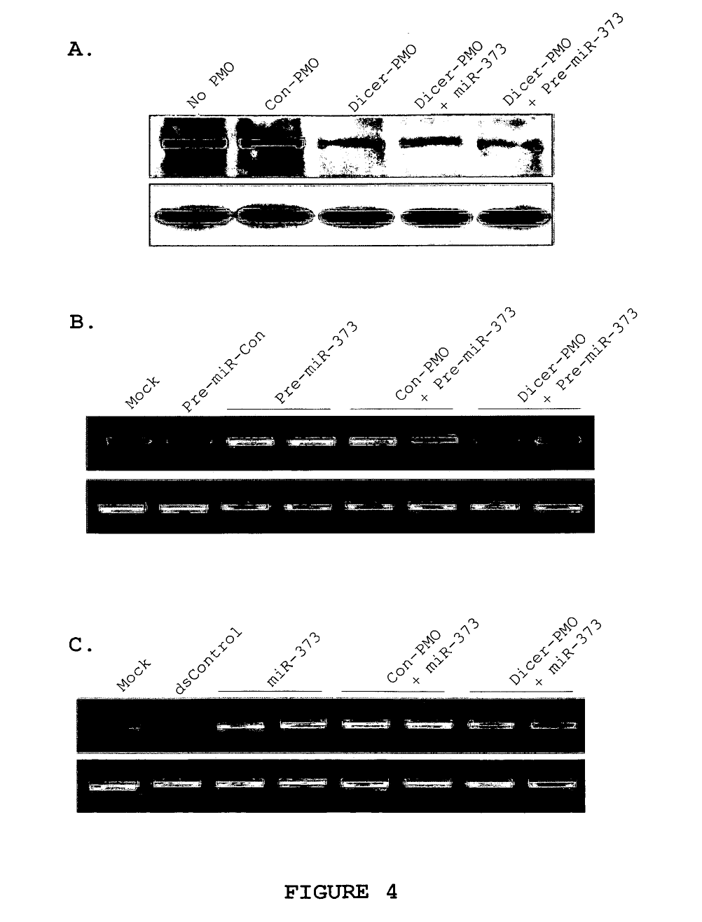

As shown in FIG. 4, panel A, immunoblot analysis demonstrates the reduction in Dicer polypeptide by antisense PMO (Dicer-PMO lane). Analysis of E-cadherin expression after treatment with the PMO shows that Dicer-PMO completely inhibited E-cadherin induction following pre-miR-373 transfection (FIG. 4, panel B). In contrast, transfected miR-373 which did not require the processing by Dicer, retained the ability to fully induce E-cadherin expression (FIG. 4, panel C). These data shows that pre-miR-373 biogenesis by Dicer produces a miR-373 able to induce E-cadherin expression, and thus miRNAs of the invention may be used to induce expression in cells containing Dicer, and miRNA may be utilized in cells without Dicer.

### Example 3

### miRNA Induces the Expression of CSDC2

miRNAs may target similar sequences in gene promoters to activate the expression of multiple genes. The open-source miRNA target prediction algorithm, miRanda (Enright et al., (2003) Genome Biol 5, R1) was used to scan 1 kb of promoter sequence from every gene in the human genome for sites highly complementary to miR-373. The results yielded over 372 genes with promoter sequences complementary to miR-373. Twelve genes were selected from the list with known function that had the greatest overall sequence complementarity to miR-373 at their respective promoter sites. The 12 representative genes are; CSDC2/PIPPIN (Cold Shock Domain Containing C2), NPM1 (nucleophosmin 1), NUPL2 (nucleoporin like 2), BFAR (bifunctional apoptosis regulator), IRAK3 (interleukin-1 receptor-associated kinase 3), GAA (Lysosomal alpha-glucosidase precursor), NSUN7 (NOL1/NOP1/Sun domain family member 7), RAGE (Renal tumor antigen 1), FOXP2 (Forkhead Box P2), RBKS (Ribokinase), ACOT6 (acyl-CoA thioesterase 6), SIRPD (Signal Regulatory Protein Delta).

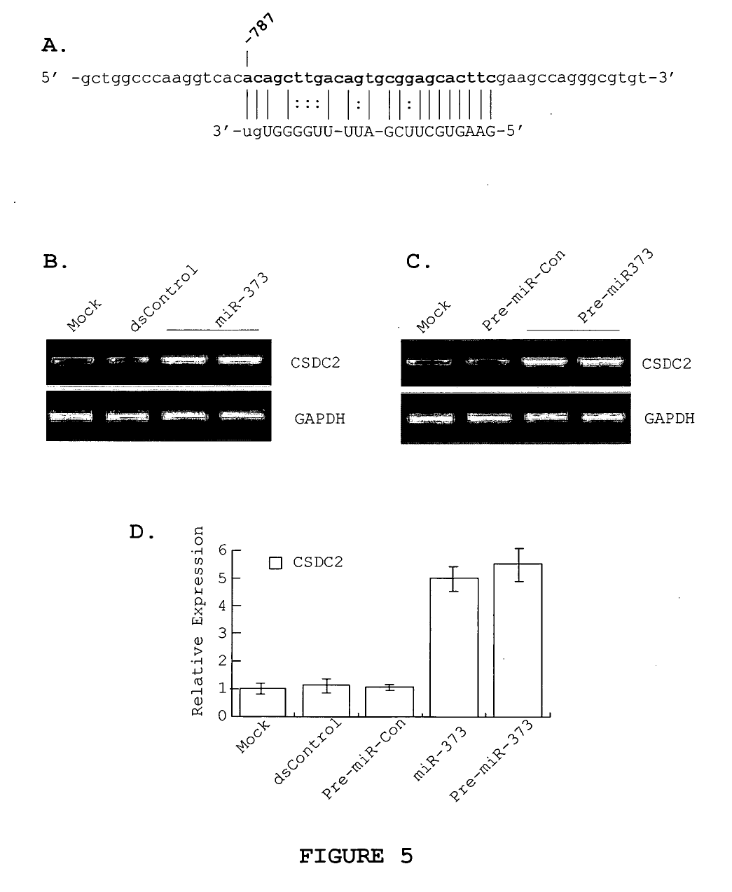

PC-3 cells were transfected with miR-373 or pre-miR-373 and the mRNA analyzed for increases in CSDC2 expression, of one of the 12 representative genes. As shown in FIG. 5, panel A, the miR-373 region of complementarity is located at minus (−)787 nucleotides in the 5′ direction relative to the transcription start site in the CSDC2 promoter. As demonstrated by FIG. 5, panel B and FIG. 5, panel C, miR-373 and pre-miR-373 readily induced the expression of CSDC2. Compared to mock transfection controls, miR-373 and pre-miR-373 induced CSDC2 expression levels by about 5-fold and about 5.2-fold, respectively (FIG. 5, panel D). These results show that similar target sequences in gene promoters may allow miRNAs of the invention to induce the expression of multiple genes. Thus, careful selection of a few miRNAs of the invention may be able to induce gene expression of multiple genes, thus saving time and materials needed to produce miRNAs for each individual promoter region.

### Example 4

### Enrichment of RNA Polymerase II at miRNA Targeted Gene Promoters

Chromatin immunoprecipitation (ChIP) assays were used to determine if enrichment of RNA polymerase II (RNApII) at targeted gene promoters was associated with miR-373 induced gene expression.

Cells were transfected with miR-373 in 100 mm dishes for 72 hours. The ChIP assays were performed using a ChIP assay kit (Upstate Biotechnology, Lake Placid, N.Y.) by following the vendor's instructions. An antibody specific to the C-terminal domain of RNApII (Millipore, Billerica, Mass.) was used to immunoprecipitate transcriptionally active regions of DNA. PCR Primers specific to E-cadherin, CSDC2 or GAPDH transcription start sites were used to amply DNA isolated in the ChIP assay. PCR was performed for 25 to 32 cycles of 95° C. for 30 seconds, 60° C. for 30 seconds, and 72° C. for 30 seconds. (See also Okino et al., (2007) Mol Pharmacol 72, 1457-65.)

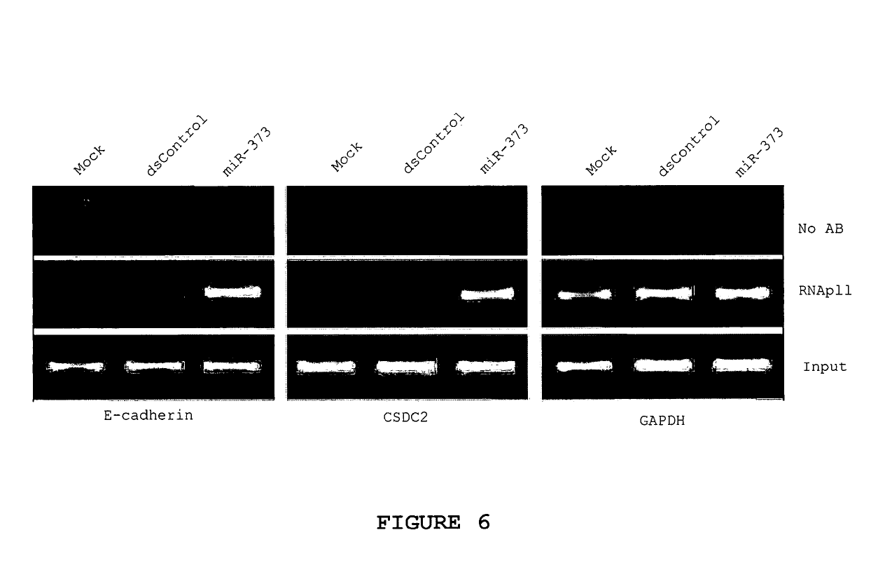

Three regions corresponding to the transcription start sites of E-cadherin, CSDC2, and GAPDH were mapped by the ChIP assay. The GAPDH promoter served as an internal control for RNApII binding. As shown in FIG. 6, mock and dsControl treatments were associated with low levels of RNApII binding; however, following miR-373 transfection, RNApII density increased at the transcription start sites of E-cadherin and CSDC2. RNApII levels at the GAPDH core promoter did not change in any treatment (FIG. 6). These results indicate that enrichment of RNApII at targeted gene promoters is associated with miR-373-induced gene expression.

### Example 5

### Sequence Specificity for Gene Induction by miRNA

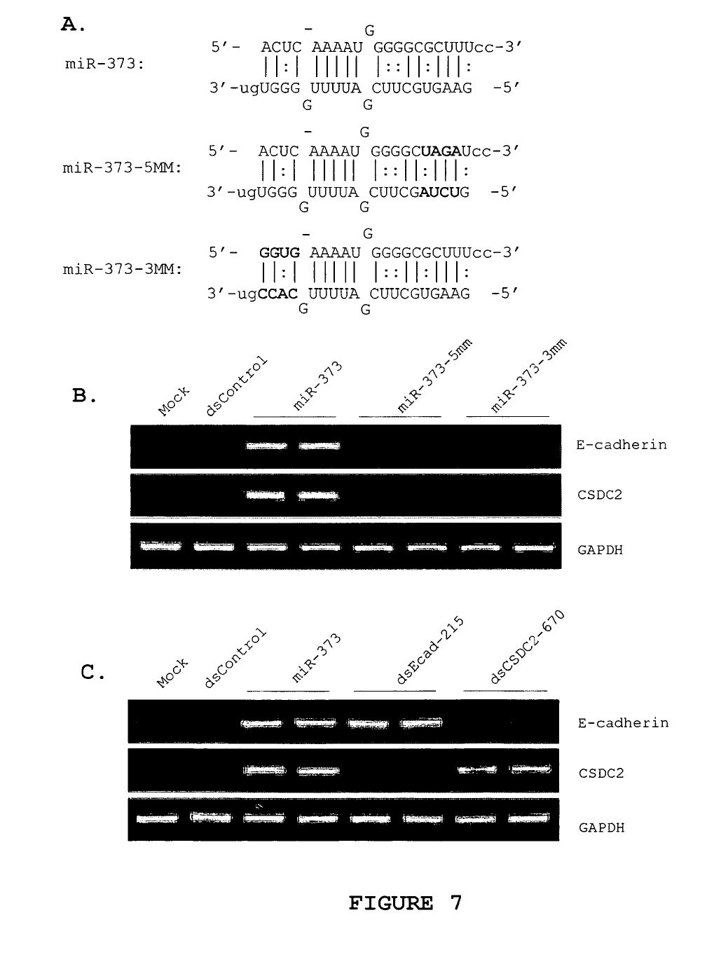

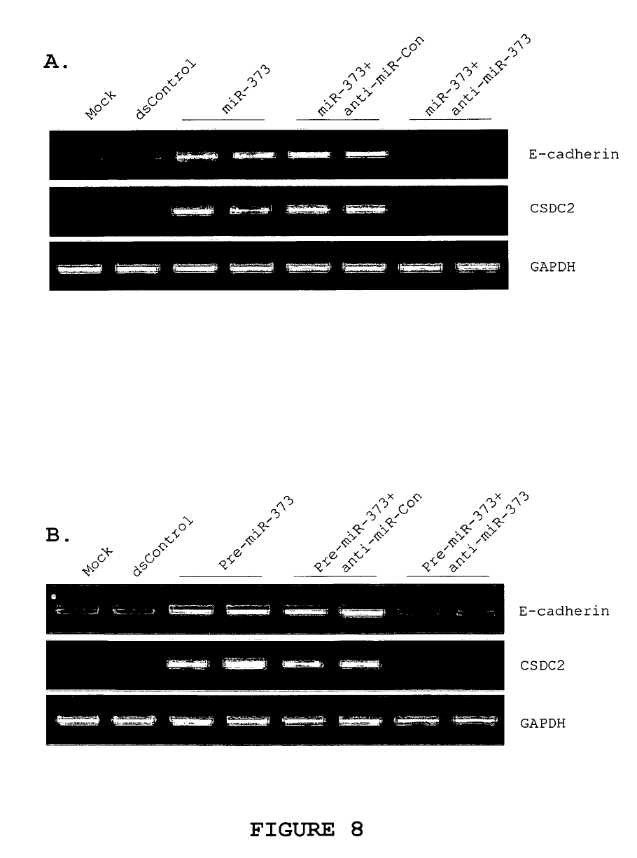

To determine the specificity of gene induction, two mutant miRNAs were synthesized. Changes to 4 bases at the 5′-end (relative to the antisense strand) or 3′-end of miR-373 resulted in mutant derivatives miR-373-5MM and miR-373-3MM, respectively (FIG. 7, panel A). As shown in FIG. 7, panel B, neither miR-373-5MM nor miR-373-3MM were capable of inducing E-cadherin or CSDC2 expression. A cotransfection with a complementary oligonucleotide (anti-miR-373) was designed specifically to bind and sequester miR-373 sequence activity. Transfection of a non-specific control oligonucleotide (anti-miR-Con) had no impact on miR-373 induced gene expression; however anti-miR-373 blocked E-cadherin and CSDC2 induction by miR-373 and pre-miR-373 (FIG. 8, panel A and FIG. 8, panel B) Taken together, these results indicate that the induction of E-cadherin and CSDC2 was specific to the sequence of miR-373.

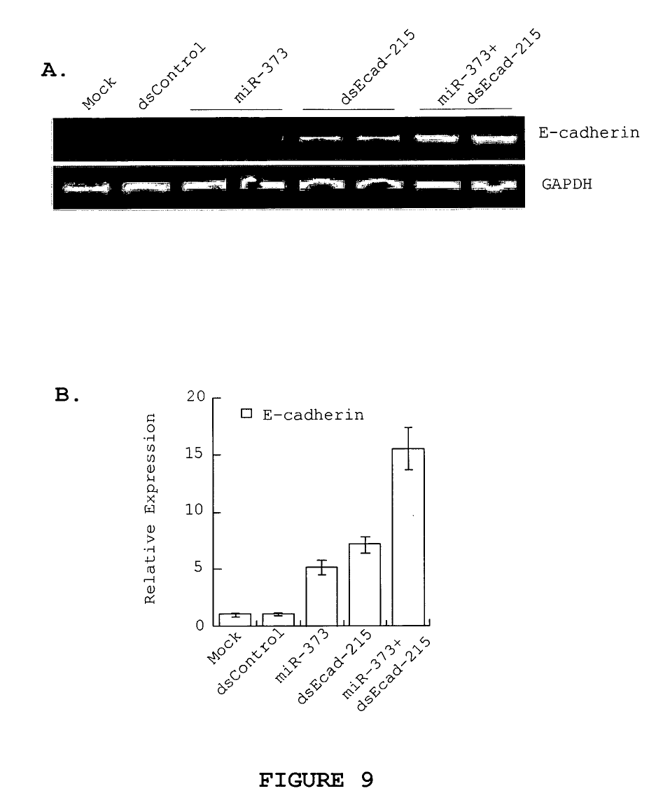

Two dsRNA molecules that specifically targeted the E-cadherin (dsEcad-215) or CSDC2 (dsCSDC2-670) promoter at different sites were synthesized to further define the specificity of the miRNAs of the invention. The inventors of the instant application had previously demonstrated that dsEcad-215 readily induced the expression of Ecadherin by targeting sequence minus(−)215 nucleotides in the 5′ direction relative to the transcription start site in the Ecadherin promoter (Li et al., (2006) Proc Natl Acad Sci USA 103, 17337-42). Using similar criteria for dsRNA design, the miRNA dsCSDC2-670 was designed to target position minus(−)670 nucleotides in the 5′ direction relative to the transcription start site in the CSDC2 promoter and induce CSDC2 expression. FIG. 7, panel C shows that transfection of miR-373 readily induced the expression of both genes, while dsEcad-215 and dsCSDC2-670 only activated the expression of E-cadherin and CSDC2, respectively. By targeting divergent sites in both promoters, gene induction was specifically limited to only target genes. These results show that activation of E-cadherin and CSDC2 occurs because miR-373 specifically targets similar sequences in the promoters of both genes. In contrast to miR-373 and dsEcad-640 co-transfections (FIG. 1, panel D and FIG. 1, panel E), the combination transfection of miR-373 and dsEcad-215 additively enhanced E-cadherin levels (FIG. 9, panel A and FIG. 9, panel B). By targeting separated sites in the E-cadherin promoter, miR-373 and dsEcad-215 both contributed to E-cadherin induction, approximately doubling it.

Thus, miRNAs of the invention may be used to further titrate the induction of gene expression. As discussed here, the use of a single miRNA (miR-373) was able to induce gene expression. The addition of another miRNA directed to a different site may prove useful in elevating the induction of gene expression to higher levels. As discussed previously, miRNA has been shown previously to suppress gene expression. The instant application shows that miRNAs of the current invention induce gene expression. It may then be possible to utilize miRNAs that are inhibitory (RNAi) to lower or silence the expression of certain genes (e.g. overexpressed genes associated with cancer) and miRNAs that activate to increase the expression of others (e.g. increased tumor suppressor genes or pro-apoptotic genes) to provide for beneficial effects, such as inducing apoptosis or reducing proliferation in cancer cells.

### Example 6

### dsRNAs Targeting the Mouse Cyclin B1 (Ccnb1) Gene Promoter Induced Ccnb1 mRNA and Protein Expression

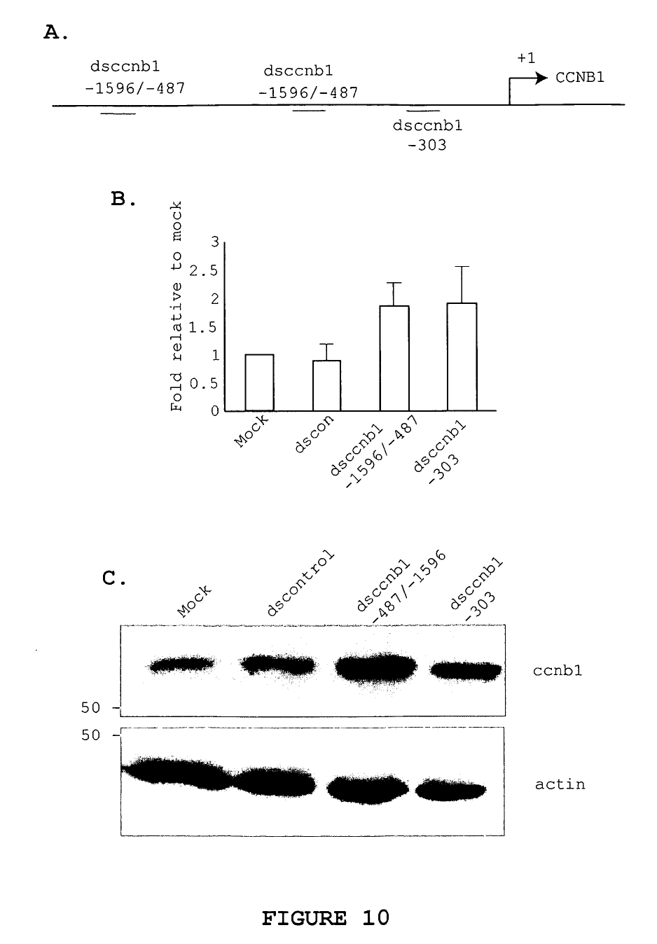

To determine whether RNAa is conserved in mouse cells, we designed two dsRNA molecules complementary to specific sites in the mouse Ccnb1 promoter; dsCcnb1-303 targeted position −303 and dsCcnb1-487/-1596 targeted a repetitive sequence located at positions −487 and −1596 relative to the Ccnb1 transcription start site (FIG. 10, panel A). Immortalized mouse NIH/3T3 embryonic fibroblast cells were transfected with either dsCcnb1-303, dsCcnb1-487/-1596, or non-specific control dsRNA (dsCon) for 72 hours and Ccnb1 expression levels were assessed by real-time PCR. Compared to mock transfections, both dsCcnb1-303 and dsCcnb1-487/-1596 caused a two-fold induction in Ccnb1 mRNA expression (FIG. 10, panel B). Induction of Ccnb1 protein levels was confirmed by immunoblot analysis (FIG. 10, panel C). Taken together, these result indicate that Ccnb1 in susceptible to RNAa in mouse cells.

### Example 7

### miR-744 and miR-1186 Targeting the Mouse Ccnb1 Gene Promoter Induced Ccnb1 Gene Expression

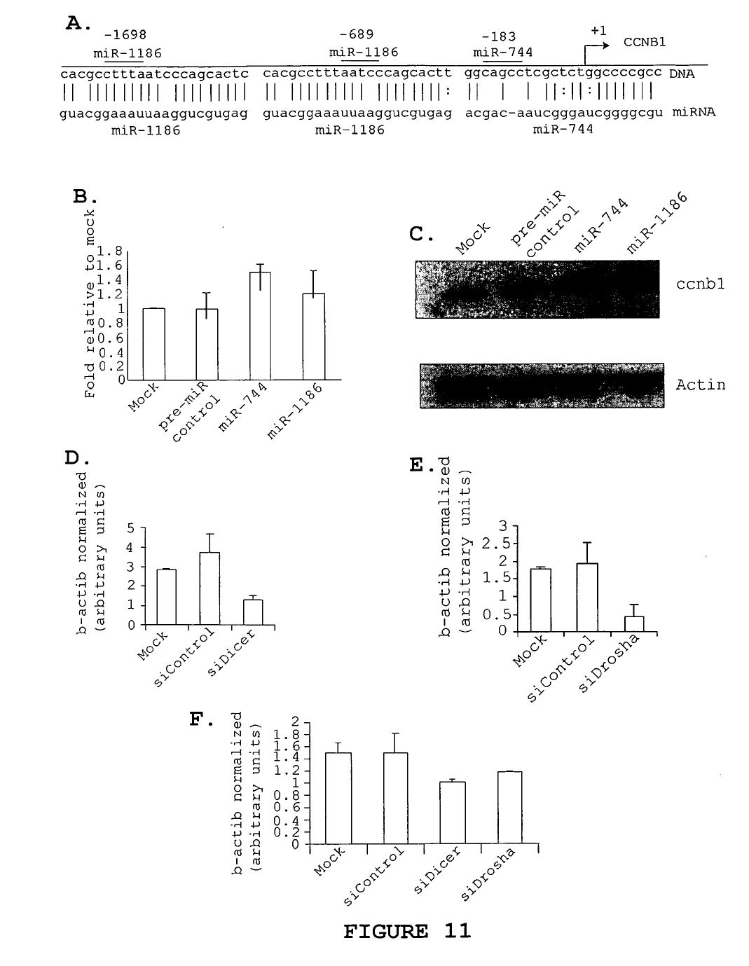

To examine whether miRNA can also direct RNAa in mouse cells, we scanned 2 kb of the Ccnb1 promoter for sites complementary to known miRNAs using the miRANDA algorithm. Scanning analysis revealed that mmu-miR-744 (miR-774) was complementary to a site located at position −183 and mmu-miR-1186 (miR-1186) had two putative target sites located at positions −698 and −1698 relative to the Ccnb1 transcription start site (FIG. 11, panel A). We transfected synthetic miR-744 and miR-1186 into NIH/3T3 cells and performed real-time PCR to assess Ccnb1 mRNA expression levels. Compared to the mock transfections, miR-744 and miR-1186 caused subtle increases in Ccnb1 mRNA levels (FIG. 11, panel B). Immunoblot analysis also confirmed increased levels in Ccnb1 protein by miR-744 and miR-1186 (FIG. 10, panel C).

Depletion of miRNA maturation genes Dicer or Drosha is known decrease total cellular levels of endogenous miRNA. To determine if endogenous miRNA may be positively regulating Ccnb1 expression, we knocked down Dicer and Drosha by sRNA and evaluated Ccnb1 expression levels. Knockdown efficiency of Dicer and Drosha was evaluated by real-time PCR (FIG. 11, panels D and E). In the presence of Dicer or Drosha sRNA, Ccnb1 levels were reduced by ˜20-30% compared to mock transfections (FIG. 11, panel F). The data shows that endogenous miRNA is positively regulating Ccnb1 gene expression.

### Example 8

### Ago1 Mediates Transcriptional Activation of Ccnb1

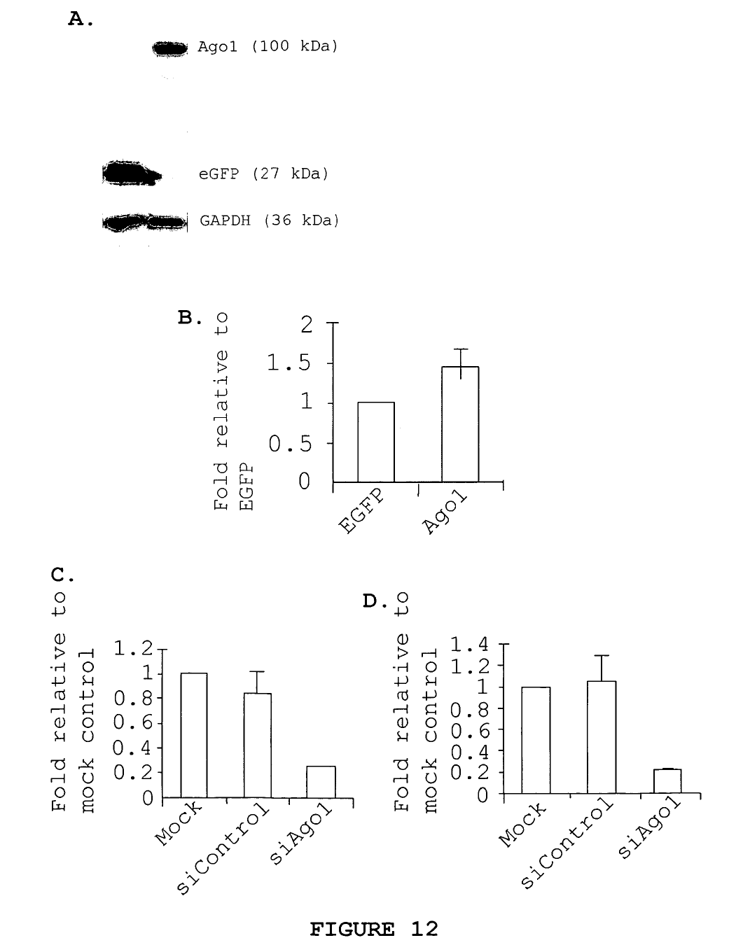

We have previously shown that the Argonaute (Ago) proteins, in particular Ago2, are involved in RNAa mediated by synthetic dsRNAs (Li et al., PNAS (2006) 103: 17337-17342). While Ago2 is known to associate with synthetic dsRNAs, such as siRNAs, to facilitate activity, Ago1 has been shown to predominantly interact with miRNAs. Furthermore, Ago1 has been shown to direct transcriptional gene silencing (TGS) by dsRNA, including miRNA, at gene promoters. We therefore decided to determine if Ago1 was also involved in miRNA-mediated gene activation. We cloned Ago1 cDNA into a mammalian expression vector downstream of a HA-epitope tag (HA-Ago1) and established a stable cell line overexpressing HA-Ago1 from NIH/3T3 cells (Ago1-NIH/3T3, FIG. 12, panel A). As a control, we also established stable NIH/3T3 cells overexpressing HA-tagged EGFP(HA-EGFP). As shown in FIG. 12, panel B, HA-Ago1 overexpression increased Ccnb1 levels by ˜1.5-fold as compared to the control HA-EGFP cell line. Conversely, knockdown of Ago1 by sRNA (FIG. 12, panel C) caused significant downregulation of Ccnb1 (FIG. 12, panel D) in NIH/3T3 cells. These results show that Ago1 positively regulates Ccnb1 expression in NIH/3T3 cells.

### Example 9

### Ago1 Associates with the Proximal Promoter Region of Ccnb1

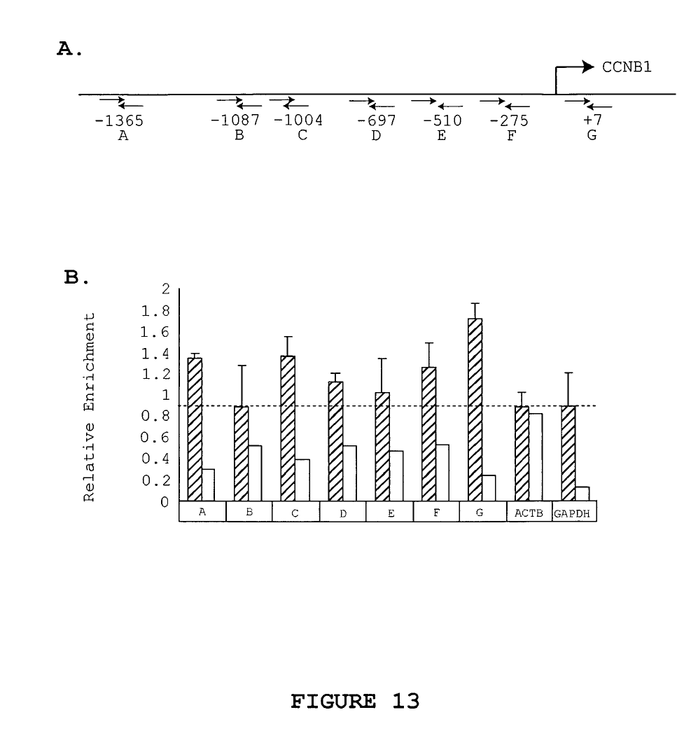

To determine if Ago1 is associated with the Ccnb1 promoter in NIH/3T3 cells, we performed chromatin immunoprecipitation (ChIP) experiments in the HA-Ago1 stable cell line utilizing an antibody specific to the HA-epitope tag. As a negative control, we also performed ChIP assays with the HA-specific antibody in the HA-EGFP cell line. Seven pairs of primers corresponding to select regions in the Ccnb1 promoter were used to quantify Ago1 enrichment by real-time PCR (FIG. 13, panel A). Data was normalized to amplified signal in INPUT samples. Fold enrichment was determined by dividing the normalized signal acquired in the presence of the HA antibody (+HA) by the background levels amplified in the no antibody control (—HA). Intragenic regions corresponding to ACTB and GAPDH, anticipated to be devoid of Ago1, were also evaluated as negative controls for signal enrichment. As shown in FIG. 13, panel B, we detected ˜1.5-2 fold enrichment of Ago1 between the −275 to +3 region relative to the Ccnb1 transcriptional start site. No enrichment in signal was observed in HA-EGFP samples at the select regions nor was Ago1 enrichment found at the intragenic ACTB and GAPDH sites. These results indicate that Ago1 is associated with the Ccnb1 promoter in NIH/3T3 cells, which correlates to Ccnb1 gene activation. This data provides further evidence that RNAa is an endogenous cellular mechanism facilitated by miRNA that can be exploited to enhance gene expression by introducing cognate promoter miRNA into a cell.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method to increase expression of a gene selected from the group consisting of; CSDC2, NPM1, NUPL2, BFAR, IRAK3, GAA, NSUN7, RAGE, FOXP2, RBKS, ACOT6, SIRPD, and CCNB1 comprising:
introducing a miRNA molecule into a mammalian cell in an amount sufficient to increase expression of the gene, wherein the miRNA molecule comprises a ribonucleic acid strand comprising:
a 5′ region of complementarity to a non-coding sequence of the gene and
a 3′ terminal region of at least one nucleotide non-complementary to the non-coding sequence,
wherein the introducing results in an increase in expression of the gene.

2. The method of claim 1, wherein the miRNA is a pre-miRNA.

3. The method of claim 1, wherein the miRNA molecule is introduced into the mammalian cell by expression from a nucleic acid vector.

4. The method of claim 1, wherein the region of complementarity comprises between about 14 to about 30 base pairs.

5. The method of claim 1, wherein the region of complementarity comprises between about 20 to about 25 base pairs.

6. The method of claim 1, wherein the gene is CSDC2.

7. The method of claim 1, wherein the ribonucleic acid strand comprises the sequence of CSDC2-670 (SEQ ID NO:11).

8. A method of decreasing proliferation of a cell comprising:
administering an effective amount of a miRNA molecule, wherein the miRNA molecule comprises a ribonucleic acid strand comprising,
a 5′ region of complementarity to a non-coding sequence of a gene, wherein the gene encodes a polypeptide that inhibits cellular proliferation, and
a 3′ terminal region of at least one nucleotide non-complementary to the non-coding sequence,
wherein the administering provides for an increase in expression of the polypeptide and a decrease in cellular proliferation.

9. The method of claim 8, wherein the miRNA is a pre-miRNA.

10. The method of claim 8, wherein the miRNA molecule is introduced into the mammalian cell by expression from a nucleic acid vector.

11. The method of claim 8, wherein the polypeptide is a tumor suppressor.

12. The method of claim 8, wherein the region of complementarity comprises between about 14 to about 30 base pairs

13. The method of claim 8, wherein the region of complementarity comprises between about 19 to about 25 base pairs.

14. A method of increasing proliferation of a cell comprising:
administering an effective amount of a miRNA molecule, wherein the miRNA molecule comprises a ribonucleic acid strand comprising
a 5′ region of complementarity to a non-coding sequence of a gene, wherein the gene encodes a polypeptide that increases cellular proliferation, and
a 3′ terminal region of at least one nucleotide non-complementary to the non-coding sequence,
wherein the administering provides for an increase in expression of the polypeptide and an increase in cellular proliferation.

15. The method of claim 14, wherein the miRNA is a pre-miRNA.

16. The method of claim 14, wherein the miRNA molecule is introduced into the mammalian cell by expression from a nucleic acid vector.

17. The method of claim 14, wherein the polypeptide is a growth factor.

18. The method of claim 14, wherein the region of complementarity comprises between about 14 to about 30 base pairs

19. The method of claim 14, wherein the region of complementarity comprises between about 19 to about 25 base pairs.

20. A method of increasing apoptosis comprising:
administering an effective amount of a miRNA molecule, wherein the miRNA molecule comprises a ribonucleic acid strand comprising,
a 5′ region of complementarity to a non-coding sequence of a gene, wherein the gene encodes a pro-apoptotic polypeptide, and
a 3′ terminal region of at least one nucleotide non-complementary to the non-coding sequence,
wherein the administering provides for an increase in expression of the polypeptide and an increase in apoptosis.

21. The method of claim 20, wherein the miRNA is a pre-miRNA.

22. The method of claim 20, wherein the miRNA molecule is introduced into the mammalian cell by expression from a nucleic acid vector.

23. The method of claim 20, wherein the region of complementarity comprises between about 14 to about 30 base pairs.

24. The method of claim 20, wherein the region of complementarity comprises between about 19 to about 25 base pairs.

25. The method of claim 20, wherein the gene encoding the pro-apoptotic polypeptide is selected from the group consisting of: Bax, SMAC, Bak, Diva, Bcl-Xs, Bik, Bim, Bad, Bid, Noxa, BID, PUMA and Egl-1.

26. An isolated composition comprising,
a miRNA molecule comprising a first ribonucleic acid strand comprising a region of complementarity to a non-coding nucleic acid sequence of a CSDC2 gene sufficient to activate transcription of the CSDC2 gene.

27. The composition of claim 26, wherein the miRNA is a pre-miRNA.

28. The composition of claim 26, wherein the miRNA molecule is encoded on a nucleic acid vector.

29. The composition of claim 26, wherein the ribonucleotide strand comprises a region of non-complimentary to the non-coding nucleic acid sequence of at least one nucleotide at a 3′ terminus.

30. The composition of claim 26, wherein the region of complementarity comprises between about 14 to about 30 base pairs.

31. The composition of claim 26, wherein the region of complementarity comprises between about 19 to about 25 base pairs.

32. The composition of claim 26, wherein the ribonucleic acid strand comprises the sequence of dsCSDC2-670 (SEQ ID NO:11).

33. A kit comprising,
a miRNA molecule comprising a first ribonucleic acid strand comprising a region of complementarity to a non-coding nucleic acid sequence of an E-cadherin gene sufficient to activate transcription of the E-cadherin gene.

34. The kit of claim 33, further comprising at least one other miRNA molecule comprising a first ribonucleic acid strand comprising a region of complementarity to a non-coding nucleic acid sequence of an E-cadherin gene sufficient to increase transcription of the activated E-cadherin gene.

35. The kit of claim 33, wherein the ribonucleic acid strand consists of the pre-miRNA of SEQ ID NO 14.

36. The kit of claim 33, wherein the miRNA is encoded on a nucleic acid vector.

37. The kit of claim 33, wherein the ribonucleic acid strand consists of the sequence of dsEcad-640 (SEQ ID NO:5).

38. The kit of claim 33, wherein the ribonucleic acid strand consists of the sequence of dsEcad-215 (SEQ ID NO:13).

39. The kit of claim 33, wherein the kit further comprises at least one of a pharmaceutically acceptable carrier, a pharmaceutically acceptable diluent, a pharmaceutically acceptable excipient and a pharmaceutically acceptable adjuvant.

40. A kit comprising,
a miRNA molecule comprising a ribonucleic acid strand comprising a region of complementarity to a non-coding nucleic acid sequence of a CSDC2 gene sufficient to activate transcription of the CSDC2 gene.

41. The kit of claim 40, wherein the miRNA molecule is a pre-miRNA molecule.

42. The kit of claim 40, wherein the miRNA molecule is encoded on a nucleic acid vector.

43. The kit of claim 40, wherein the ribonucleotide strand comprises a region of non-complimentary to the non-coding nucleic acid sequence of at least one nucleotide at a 3′ terminus.

44. The kit of claim 40, wherein the region of complementarity comprises between about 14 to about 30 base pairs.

45. The kit of claim 40, wherein the region of complementarity comprises between about 19 to about 25 base pairs.

46. The kit of claim 40, wherein the ribonucleic acid strand comprises the sequence of dsCSDC2-670 (SEQ ID NO:11).


SEQUENCE LISTING (28 sequences)

SEQ ID NO: 1 - DNA, 55 bp, Homo sapiens
cagtggctctatggctcacacctgaaatcctagcactttgggaggccaaggcagg

SEQ ID NO: 2 - RNA, 22 nt, Artificial Sequence
  Synthetic oligonucleotide
acucaaaaugggggcgcuuucc

SEQ ID NO: 3 - RNA, 23 nt, Artificial Sequence
  Synthetic oligonucleotide
gaagugcuucgauuuuggggugu

SEQ ID NO: 4 - DNA, 21 bp, Artificial Sequence
  Combined DNA/RNA synthetic oligonucleotide
ccugaaauccuagcacuuutt

SEQ ID NO: 5 - DNA, 21 bp, Artificial Sequence
  Combined DNA/RNA synthetic oligonucleotide
aaagugcuaggcuuucaggtt

SEQ ID NO: 6 - RNA, 22 nt, Artificial Sequence
  Synthetic oligonucleotide
acucaaaaugggggcuagaucc

SEQ ID NO: 7 - RNA, 23 nt, Artificial Sequence
  Synthetic oligonucleotide
gucuagcuucgauuuuggggugu

SEQ ID NO: 8 - RNA, 22 nt, Artificial Sequence
  Synthetic oligonucleotide
ggugaaaaugggggcgcuuucc

SEQ ID NO: 9 - RNA, 23 nt, Artificial Sequence
  Synthetic oligonucleotide
gaagugcuucgauuuugcaccgu

SEQ ID NO: 10 - DNA, 21 bp, Artificial Sequence
  Combined DNA/RNA synthetic oligonucleotide
guucaccugugcaccuucatt

SEQ ID NO: 11 - DNA, 21 bp, Artificial Sequence
  Combined DNA/RNA synthetic oligonucleotide
ugaaggugcacaggugaactt

SEQ ID NO: 12 - DNA, 21 bp, Artificial Sequence
  Combined DNA/RNA synthetic oligonucleotide
aaccgugcaggucccauaatt

SEQ ID NO: 13 - DNA, 21 bp, Artificial Sequence
  Combined DNA/RNA synthetic oligonucleotide
uuaugggaccugcacgguutt

SEQ ID NO: 14 - RNA, 61 nt, Artificial Sequence
  Synthetic oligonucleotide
acucaaaaugggggcgcuuuccuuuuugucuguacugggaagugcuucgauuuuggggugu

SEQ ID NO: 15 - DNA, 20 bp, Artificial Sequence
  Synthetic oligonucleotide
cctgggactccacctacaga

SEQ ID NO: 16 - DNA, 20 bp, Artificial Sequence
  Synthetic oligonucleotide
ggatgacacagcgtgagaga

SEQ ID NO: 17 - DNA, 20 bp, Artificial Sequence
  Synthetic oligonucleotide
gttcaagggcgtctgtaagc

SEQ ID NO: 18 - DNA, 20 bp, Artificial Sequence
  Synthetic oligonucleotide
agctgagtgagcaccacctc

SEQ ID NO: 19 - DNA, 18 bp, Artificial Sequence
  Synthetic oligonucleotide
tcccatcaccatcttcca

SEQ ID NO: 20 - DNA, 18 bp, Artificial Sequence
  Synthetic oligonucleotide
catcacgccacagtttcc

SEQ ID NO: 21 - DNA, 25 bp, Artificial Sequence
  Synthetic oligonucleotide
agcagggcttttcattcatccagtg

SEQ ID NO: 22 - DNA, 23 bp, Artificial Sequence
  Synthetic oligonucleotide
ataacccacctagaccctagcaa

SEQ ID NO: 23 - DNA, 21 bp, Artificial Sequence
  Synthetic oligonucleotide
ctcacaggtgctttgcagttc

SEQ ID NO: 24 - DNA, 24 bp, Artificial Sequence
  Synthetic oligonucleotide
aagcagggactacaaattctcatc

SEQ ID NO: 25 - DNA, 22 bp, Artificial Sequence
  Synthetic oligonucleotide
ctctgtctctctctggctcgtg

SEQ ID NO: 26 - DNA, 25 bp, Artificial Sequence
  Synthetic oligonucleotide
tactagcggttttacgggcgcacgt

SEQ ID NO: 27 - DNA, 25 bp, Artificial Sequence
  Combined DNA/RNA synthetic oligonucleotide
tcgaacaggaggagcagagagcgaa

SEQ ID NO: 28 - DNA, 56 bp, Homo sapiens
gctggcccaaggtcacacagcttgacagtgcggagcacttcgaagccagggcgtgt